(19) United States
(12) Patent Application Publication
(10) Pub. No.: US 2023/0000766 A1
(43) Pub. Date: Jan. 5, 2023

(54) PROCESS FOR THE PREPARATION OF DISPERSIONS COMPRISING INHALABLE IMMUNOSUPPRESSIVE ACTIVE INGREDIENTS

(71) Applicant: Zambon S.p.A., Milan (IT)

(72) Inventor: Oliver Denk, Muensing (DE)

(21) Appl. No.: 17/778,007

(22) PCT Filed: Dec. 21, 2020
(86) PCT No.: PCT/EP2020/087471

§ 371 Date: May 19, 2022

(30) Foreign Application Priority Data
Dec. 23, 2019 (EP) 19219340.7

(51) Int. Cl. A61K 9/00; A61K 9/127; A61K 38/13; A61K 9/19

(52) U.S. Cl. CPC A61K 9/0073; A61K 9/1277; A61K 38/13; A61K 9/19

(57) ABSTRACT

The present invention relates to a process for the preparation of a dispersion comprising an inhalable immunosuppressive macrocyclic active ingredient in liposomally solubilized form in an aqueous carrier liquid, the process comprising the steps of a) providing a mixture comprising —the inhalable immunosuppressive macrocyclic active ingredient; —a membrane-forming substance selected from the group of phospholipids; —a solubility-enhancing substance selected from the group of non-ionic surfactants; —optionally one or more excipients; and —the aqueous carrier liquid; b) dispersing the mixture as provided in step a) to form an intermediate aqueous dispersion comprising the inhalable immunosuppressive macrocyclic active ingredient in the aqueous carrier liquid; and c) homogenizing the intermediate aqueous dispersion as formed in step b) to form the dispersion comprising the inhalable immunosuppressive macrocyclic active ingredient in liposomally solubilized form.

### FIELD

The present invention relates to a process for the preparation of a dispersion comprising an inhalable immunosuppressive macrocyclic active ingredient in liposomally solubilized form in an aqueous carrier liquid. Specifically, the present invention relates to a process for the preparation of dispersions comprising cyclosporine A in liposomally solubilized form. Furthermore, the present invention relates to a process for the preparation of lyophilized pharmaceutical compositions comprising an inhalable immunosuppressive active ingredient as well as to lyophilized pharmaceutical compositions obtainable by such a process.

### BACKGROUND

Cyclosporine (CsA or ‘ciclosporin’ as used herein synonymously) is a cyclic oligopeptide with immunosuppressive and calcineurin inhibitory activity. It is characterised by a selective and reversible mechanism of immunosuppression by blocking the activation of T-lymphocytes by the production of certain cytokines which are involved in the regulation of these T-cells. This involves, in particular, the inhibition of the synthesis of interleukin-2 which, at the same time, suppresses the proliferation of cytotoxic T-lymphocytes which are responsible, for example, for the rejection of extraneous tissues. Cyclosporine acts intracellularly by binding to the so-called cyclophilines or immunophilines which belong to the family of proteins which bind cyclosporine with high affinity. The complex of cyclosporine and cyclophilin subsequently blocks the serine-threonine-phosphatase-calcineurin. Its activity state in turn controls the activation of transcription factors such as NF-KappaB or NFATp/c which play a decisive role in the activation of various cytokine genes including interleukin-2. This results in the arrest of the immunocompetent lymphocytes during the G0 or G1 phase of the cellular cycle since the proteins which are essential for cell division such as interleukin-2 can no longer be produced. T-helper cells which increase the activity of cytotoxic T-cells which are responsible for rejection are the preferred site of attack for cyclosporine. Furthermore, cyclosporine inhibits the synthesis and release of further lymphokines which are responsible for the proliferation of mature cytotoxic T-lymphocytes and for other functions of the lymphocytes. The ability of cyclosporine to block interleukin-2 is critical for its clinical efficacy: Transplant recipients which tolerate their transplants well are characterised by a low production of interleukin-2. Patients with manifest rejection reactions, on the contrary, show no inhibition of interleukin-2 production.

The first and so far only cyclosporine which has been placed on the market (in the 1980s) is cyclosporine A (CsA). CsA is defined chemically as cyclo-[[(E)-(2S,3R,4R)-3-hydroxy-4-methyl-2-(methylamino)-6-octenoyl]-L-2-aminobutyryl-N-methylglycyl-N-methyl-L-leucyl-L-valyl-N-methyl-L-leucyl-L-alanyl-D-alanyl-N-methyl-L-leucyl-N-methyl-L- leucyl-N-methyl-L-valyl]. Its availability initiated a new era in transplant medicine because, with its help, the proportion of transplanted organs which remain functional in the long term, could be increased substantially. The first cyclosporine medicament (Sandimmun® of Sandoz) could already increase the success rate in kidney transplantations by a factor of about 2. A new oral preparation of cyclosporine (Neoral® of Sandoz, later Novartis) with higher and more reliable bioavailability allowed better dosing and further increase of the success rate since the 1990s. Despite some new developments of active agents, CsA is still a frequently used agent in transplantation medicine.

Today, lung transplantations can, in principle, also be carried out successfully if patients are treated with CsA. Since the introduction of this active agent in clinical therapy, the number of lung transplantations carried out worldwide has increased dramatically. This is true for both, the transplantation of a single lung as well as the transplantation of both lungs. Lung transplantations are normally contemplated in the case of patients with a final-staged lung disease where medicinal therapy has failed and life expectancy is short due to the disease. Transplantations of a single lung are indicated, for example, in the case of certain forms of emphysema and fibrosis, such as idiopathic pulmonary fibrosis. Both lungs are transplanted in cases of cystic fibrosis (mucoviscidosis), primary pulmonary hypertension, emphysema with global insufficiency, frequent serious infections as well as idiopathic pulmonary fibrosis with complication by repeated infections. In the case of a successful lung transplantation, the patients' quality of life can be increased again to an almost normal level. However, contrary to heart, kidney and liver transplantations, the survival times after lung transplantations are still relatively short and amount to an average of only 5 years. This might be due, amongst other things, to the fact that the active agent cyclosporine cannot be effectively dosed with all patients due to systemic side effects such as renal dysfunction, increased serum levels of creatinine and urea, renal damage with structural changes, for example, interstitial fibrosis, increased serum levels of bilirubine and liver enzymes, hypertrichiosis, tremor, fatigue, headache, gingivitis hypertrophicans, gastrointestinal complains like anorexia, abdominal pain, nausea, vomiting, diarrhoea, gastritis, gastroenteritis, paraesthesia, stinging sensations in the hands and feet, arterial hypertension, increased blood fat levels, acne, rashes, allergic skin reactions, hyperglycaemia, anaemia, hyperuricaemia, gout, increasing body weight, oedemas, stomach ulcers, convulsions, menstrual disorders, hyperkalaemia, hypomagnesaemia, hot flushes, erythema, itching, muscular cramps, muscular pain, myopathy, etc.

Therefore, it would be desirable, if, for example, after a lung transplantation or in cases of certain other indications, CsA could be administered in a targeted and tissue specific fashion and so as to achieve only a low systemic bioavailability of the active agent in order to minimize the impact of the active agent in healthy tissue.

A suitable dosage form could also be used for the treatment and prevention of diseases such as asthma, idiopathic pulmonary fibrosis, sarcoidosis, alveolitis and parenchymal lung diseases (see: Drugs for the treatment of respiratory diseases, edited by Domenico Spina, Clive p. Page et. al., Cambridge University Press, 2003, ISBN 0521773210). New therapeutic aspects also result for the topical treatment of possible autoimmune included diseases such as neurodermatitis, psoriasis, unspecific eczema, skin proliferations or mutations, and for the treatment after skin transplantations. An interesting area of application is in the field of ophthalmology, for example, for the treatment after corneal transplants, of keratoconjunctivitis or other infectious eye diseases which respond partly insufficiently to anti-inflammatory therapy, for example with steroids. It is also useful for the treatment of keratitis in animals, such as dogs.

Attempts have been made to administer cyclosporine locally, for example, in the form of oily eye drops at 1% and 2% (formulation according to the German codex of medicines using refined peanut oil as solubilizer) or as an aerosol. However, this approach normally fails, mainly due to the very low aqueous solubility of the active agent which renders efficient administration considerably difficult. Thus, in the case of pulmonary application, certain adjuvants for solubilization which may be used in the case of oral administration cannot be employed for lack of tolerability. For example, Sandimmun® Optoral capsules (Novartis) which contain cyclosporine A, comprise a microemulsion concentrate with ethanol, propylene glycol and significant amounts of surfactants and, therefore, constitute a formulation which, if inhaled, would cause serious toxic effects. Similarly, the Sandimmun® infusion solution concentrate (Novartis), which is available for infusion, is also not inhalable: The only adjuvants contained therein are ethanol and poly(oxyethylene)-40-castor oil. It can be used for infusion only because it is previously diluted with a 0.9% sodium chloride solution or a 5% glucose solution, at a ratio of 1:20 to 1:100. This results in large volumes which can be administered by infusion, but not by inhalation.

WO 2007/065588 A1 discloses liquid pharmaceutical compositions containing a therapeutically effective dose of a cyclosporin; an aqueous carrier liquid; a first solubilizing substance selected among the group of phospholipids; and a second solubilizing substance selected among the group of non-ionic surfactants. The disclosed composition is suitable for oral, parenteral, nasal, mucosal, topical, and particularly pulmonary application in the form of an aerosol.

WO 2016/146645A1 discloses liposomal cyclosporine formulations that preferably comprise unilamellar liposomes. The liposomes preferably have an average diameter of at most about 100 nm measured as z-average using photon correlation spectroscopy and a polydispersity index of at most about 0.5 as measured by photon correlation spectroscopy.

The formulation can be presented as a solid formulation for reconstitution with an aqueous solvent immediately before inhalation. The solid formulation can be prepared by any method suitable for removing the solvent from a liquid formulation. Preferred examples of methods for preparing such solid formulation are freeze drying and spray drying. To protect the active ingredient during the drying process, it may be useful to incorporate lyoprotective and/or bulking agents, such as a sugar or a sugar alcohol, in particular sucrose, fructose, glucose, trehalose, mannitol, sorbitol, isomalt, or xylitol. Most notably, however, the sugar is added to the preformed formulation comprising the liposomal encapsulated CsA.

With regard to the high potency of many macrocyclic immunosuppressive active ingredients, such as cyclosporine A, tacrolimus, sirolimus and/or everolimus, as well as in view of the potentially serious unwanted side effects that these potent compounds may have when not properly dosed, it is of great importance that the content of these active ingredients in pharmaceutical compositions comprising such macrocyclic immunosuppressive compounds can be precisely controlled in the manufacturing process. This is important to avoid potentially serious side effects as described above due to overdosing these compounds or to avoid lack of efficacy due to underdosing which might also lead to serious complications such as transplant rejection.

Therefore, it is of outmost importance that the content of the macrocyclic immunosuppressive active agent can de be determined and controlled in each step of the manufacturing process of a pharmaceutical composition comprising such active ingredients.

In this context, especially the very low solubility in aqueous solution of many macrocyclic immunosuppressive agents, especially of cyclosporin A and tacrolimus, is considered problematic. These compounds usually are prepared by fermentation and further purified by crystallization. The physical properties of these highly purified compounds, unfortunately, tend to change in the course of time, for example due to aggregation, agglutination or clumping processes on which larger particles are formed which may have a different, often less favorable dispersion or dissolution behavior. This, however, especially when material which has not been freshly prepared is used, may raise the risk for inaccurate concentrations of dispersed or dissolved active ingredients in the manufacturing process and in the final dosage form, accordingly.

It is therefore an object of the present invention to provide an improved process for the preparation of pharmaceutical compositions comprising an inhalable immunosuppressive active ingredient such as cyclosporine A, tacrolimus, sirolimus, everolimus or others which is, to a large extend, independent of the physical properties with which the macrocyclic immunosuppressive active ingredient is supplied and deployed.

Further objects of the present invention will become apparent from the present disclosure including the examples and claims.

### SUMMARY OF THE INVENTION

In the first aspect, the invention relates to a process for the preparation of a dispersion comprising an inhalable immunosuppressive macrocyclic active ingredient in liposomally solubilized form in an aqueous carrier liquid, the process comprising the steps of
a) providing a mixture comprising
   the inhalable immunosuppressive macrocyclic active ingredient;
   a membrane-forming substance selected from the group of phospholipids;
   a solubility-enhancing substance selected from the group of non-ionic surfactants;
   optionally one or more excipients; and
   the aqueous carrier liquid;
b) dispersing the mixture as provided in step a) to form an intermediate aqueous dispersion comprising the inhalable immunosuppressive macrocyclic active ingredient in the aqueous carrier liquid; and
c) homogenizing the intermediate aqueous dispersion as formed in step b) to form the dispersion comprising the inhalable immunosuppressive macrocyclic active ingredient in liposomally solubilized form.

In a second aspect, the present invention relates to a process for the preparation of a lyophilized pharmaceutical composition for reconstitution in an aqueous carrier liquid, the lyophilized pharmaceutical composition comprising an inhalable immunosuppressive macrocyclic active ingredient in liposomally solubilized form, wherein the process comprises the preparation of a dispersion comprising an inhalable immunosuppressive macrocyclic active ingredient in liposomally solubilized form in an aqueous carrier liquid according to the process of the first aspect of the invention; and further comprising the step of
d) removing the aqueous carrier liquid at least partially under lyophilization conditions to form the lyophilized pharmaceutical composition.

In a third aspect, the present invention provides a lyophilized pharmaceutical composition comprising an inhalable immunosuppressive macrocyclic active ingredient in liposomally solubilized form for reconstitution in an aqueous carrier liquid, wherein the composition is obtained or obtainable by a process according to the second aspect of the invention.

In a fourth aspect, the present invention relates to the lyophilized pharmaceutical composition according to the third aspect of the invention for use as a medicament for pulmonary application.

### DETAILED DESCRIPTION OF THE INVENTION

The terms “consist of”, “consists of” and “consisting of” as used herein are so-called closed language meaning that only the mentioned components are present. The terms “comprise”, “comprises” and “comprising” as used herein are so-called open language, meaning that one or more further components may or may not also be present.

The term “active pharmaceutical ingredient” (also referred to as “API” throughout this document) refers to any type of pharmaceutically active compound or derivative that is useful in the prevention, diagnosis, stabilization, treatment, or—generally speaking—management of a condition, disorder or disease.

The term “therapeutically effective amount” as used herein refers to a dose, concentration or strength which is useful for producing a desired pharmacological effect. In the context of the present invention, the term “therapeutically effective” also includes prophylactic activity. The therapeutic dose is to be defined depending on the individual case of application. Depending on the nature and severity of the disease, route of application as well as height and state of the patient, a therapeutic dose is to be determined in a way known to the skilled person.

In the context of the present invention, a “pharmaceutical composition” is a preparation of at least one API and at least one adjuvant, which, in the simplest case, can be, for example, an aqueous liquid carrier such as water or saline.

‘A’ or ‘an’ does not exclude a plurality; i.e. the singular forms ‘a’, ‘an’ and ‘the’ should be understood as to include plural referents unless the context clearly indicates or requires otherwise. In other words, all references to singular characteristics or limitations of the present disclosure shall include the corresponding plural characteristic or limitation, and vice versa, unless explicitly specified otherwise or clearly implied to the contrary by the context in which the reference is made. The terms ‘a’, ‘an’ and ‘the’ hence have the same meaning as ‘at least one’ or as ‘one or more’ unless defined otherwise. For example, reference to ‘an ingredient’ includes mixtures of ingredients, and the like.

When used herein the term ‘about’ or ‘ca.’ will compensate for variability allowed for in the pharmaceutical industry and inherent in pharmaceutical products, such as differences in content due to manufacturing variation and/or time-induced product degradation. The term allows for any variation, which in the practice of pharmaceuticals would allow the product being evaluated to be considered bioequivalent in a mammal to the recited strength of a claimed product.

‘Essentially’, ‘about’, ‘approximately’, ‘substantially” and the like in connection with an attribute or value include the exact attribute or the precise value, as well as any attribute or value typically considered to fall within a normal range or variability accepted in the technical field concerned. For example, ‘substantially free of water” means that no water is deliberately included in a formulation, but does not exclude the presence of residual moisture.

In the context of the present invention, a “colloidal aqueous solution” preferably means a solution without organic solvent consisting of mainly unilamellar liposomes having a mean diameter of at most 100 nm and/or a polydispersity index (PI) of not more than 0.50 in which the active agent is, at least predominantly, dissolved. Preferably, water, or more specifically saline is the only liquid solvent contained in the preparation. Furthermore, it is preferred that the preparation is an aqueous solution or an aqueous colloidal solution, i.e., a monophasic liquid system. Such a system is essentially free of dispersed particles having a greater than colloidal particle size. By convention, particles below about 1 μm are regarded as colloidal particles which do not constitute a separate phase and do not result in a physical phase boundary. Sometimes, particles in a size range just above 1 μm are also still considered colloidal. Preferably, however, colloidal aqueous solutions as used herein are essentially free of particles which do clearly not belong to the colloidal spectrum, i.e., for example, particles having a diameter of 1 μm or more.

According to the first aspect, the present invention provides a process for the preparation of a dispersion comprising an inhalable immunosuppressive macrocyclic active ingredient in liposomally solubilized form in an aqueous carrier liquid, the process comprising the steps of
a) providing a mixture comprising
   the inhalable immunosuppressive macrocyclic active ingredient;
   a membrane-forming substance selected from the group of phospholipids;
   a solubility-enhancing substance selected from the group of non-ionic surfactants;
   optionally one or more excipients; and
   the aqueous carrier liquid;
b) dispersing the mixture as provided in step a) to form an intermediate aqueous dispersion comprising the inhalable immunosuppressive macrocyclic active ingredient in the aqueous carrier liquid; and
c) homogenizing the intermediate aqueous dispersion as formed in step b) to form the dispersion comprising the inhalable immunosuppressive macrocyclic active ingredient in liposomally solubilized form.

The process according to this first aspect of the present invention is suitable for the preparation of a dispersion comprising an inhalable immunosuppressive macrocyclic active ingredient in liposomally solubilized form. The term “dispersion” as used herein is to be understood in a broad sense and means, unless otherwise specified, a system, in which distributed particles as a dispersed phase are dispersed in a continuous phase, specifically in a liquid continuous phase, especially in an aqueous liquid as the continuous phase. An ‘Inhalable immunosuppressive macrocyclic active ingredient’ as used in the context of the present invention is to be interpreted in a broad sense and comprises immunosuppressive macrocyclic active ingredients that can be administered to human or animal, preferably to a human or warm-blooded animal, specifically to a human, by inhalation, whereas during such inhalation the immunosuppressive macrocyclic active ingredient or API is transported to at least a part or portion of the respiratory system of said animal or human, specifically to the lungs of said animal or human.

In the context of the present invention an immunosuppressive macrocyclic active ingredient may be, for example, cyclosporin A (hereinafter also referred to as ‘CsA’), tacrolimus, sirolimus and/or everolimus, specifically however, cyclosporin A and/or tacrolimus, more specifically cyclosporin A, wherein cyclosporine A is defined chemically as cyclo-[[(E)-(2S,3R,4R)-3-hydroxy-4-methyl-2-(methylamino)-6-octenoyl]-L-2-aminobutyryl-N-methylglycyl-N-methyl-L-leucyl-L-valyl-N-methyl-L-leucyl-L-alanyl-D-alanyl-N-methyl-L-leucyl-N-methyl-L-leucyl-N-methyl-L-valyl] (CAS number 59865-13-3) and is a cyclic peptide with immunosuppressive activity.

In the context of the present invention, cyclosporin A may be used in any quality or of any origin that is suitable for the preparation of a pharmaceutical composition. For example, cyclosporin A may be prepared by standard fermentation techniques and may, in specific embodiments, be used in any suitable form, such as in any particle size or powder form in the context of the process according to the present invention.

The process according to the present invention allows to prepare the inhalable immunosuppressive macrocyclic ingredient such as cyclosporine A, tacrolimus, sirolimus and/or everolimus or a mixture comprising one or more of these compounds, specifically cyclosporin A, in liposomally solubilized form. The term “liposomally solubilized form” as used herein means that the at least one inhalable immunosuppressive macrocyclic ingredient as described above, specifically CsA, is incorporated or intercalated in the liposome-forming structures to be formed by the other ingredients, specifically by the membrane-forming substance selected from the group of phospholipids and by a solubility-enhancing substance selected from the group of non-ionic surfactants as described in further detail below. The liposome-forming structures as referred to herein, however, may or may not have a continuous or closed bilayer membrane. In specific embodiments, the liposome-forming structures may at least be partly present in unilamellar form or, preferably, may predominantly be present in unilamellar form. The term “unilamellar” as used herein means that the corresponding liposome-forming structures only comprise a single layer formed by a single lipid bilayer membrane and not a plurality of lipid bilayer membranes in a layered arrangement.

According to step a) of the process of to the present invention a mixture is provided comprising, as a first ingredient, the at least one inhalable immunosuppressive macrocyclic active ingredient as described above, specifically CsA. As the second ingredient, the mixture to be provided according to step a) comprises a membrane-forming substance selected from the group of phospholipids, or a mixture of two or more different membrane forming substances selected from the group of phospholipids.

The term “membrane-forming substance” as used herein means that the substance is capable of forming a lipid bilayer membrane by self-assembly in an aqueous carrier liquid, such as water or saline and/or is capable of forming liposomes in an aqueous carrier liquid under conditions or circumstances as described in further detail below.

Preferred phospholipids comprised by the liposome forming structures of the present invention are, in particular, mixtures of natural or enriched phospholipids, for example, lecithins such as the commercially available Phospholipon® G90, 100, or Lipoid 90, S 100. Accordingly, in specific embodiments, the membrane-forming substance selected from the group of phospholipids is a mixture of natural phospholipids.

Phospholipids are amphiphilic lipids which contain phosphorus. Known also as phosphatides, they play an important role in nature, especially as the double layer forming constituents of biological membranes and frequently used for pharmaceutical purposes are those phospholipids which are chemically derived from phosphatidic acid. The latter is a (usually doubly) acylated glycerol-3-phosphate in which the fatty acid residues may be of different lengths. The derivatives of phosphatidic acids are, for example, the phosphocholines or phosphatidylcholines, in which the phosphate group is additionally esterified with choline, as well as phosphatidylethanolamine, phosphatidylinositols etc. Lecithins are natural mixtures of various phospholipids which usually contain a high proportion of phosphatidylcholines. Preferred phospholipids according to the invention are lecithins as well as pure or enriched phosphatidylcholines such as dimyristoylphospatidylcholine, di-palmitoyl-phosphatidylcholine and distearoylphosphatidylcholine.

In further specific embodiments, the membrane-forming substance selected from the group of phospholipids to be provided in the mixture of step a) of the present invention is a lecithin containing unsaturated fatty acid residues. In yet further specific embodiments, the membrane-forming substance selected from the group of phospholipids is a lecithin selected from the group consisting of soybean lecithin, Lipoid 90, Lipoid S75, Lipoid S100, Phospholipon® G90, Phospholipon®100 or a comparable lecithin. In further specific embodiments, the membrane-forming substance selected from the group of phospholipids is selected from Lipoid S100, Lipoid S75, particularly Lipoid S100.

The third ingredient of the mixture to be provided according to step a) of the process of the present invention is a solubility-enhancing substance selected from the group of non-ionic surfactants or a mixture of two or more different solubility-enhancing substances selected from the group of non-ionic surfactants. Non-ionic surfactants have—as other surfactants—at least one rather hydrophilic and at least one rather lipophilic molecular region. There are monomeric, low molecular weight non-ionic surfactants and non-ionic surfactants having an oligomeric or polymeric structure. Examples of suitable non-ionic surfactants suitable as solubility-enhancing substances according to be comprised by the mixture of step a) of the present invention comprise polyoxyethylene alkyl ethers, polyoxyethylene sorbitan fatty acid esters such as, for example, polyoxyethylene sorbitan oleate, sorbitan fatty acid esters, poloxamers, vitamin E-TPGS (D-α-tocopheryl polyethylene glycol 1000 succinate) and tyloxapol.

In specific embodiments, the solubility-enhancing substance selected from the group of non-ionic surfactants may be selected from the group of polysorbates and vitamin E-TPGS, preferably is selected from the group of polysorbates. In a particularly preferred embodiment, the solubility-enhancing substance selected from the group of non-ionic surfactants is polysorbate 80.

As an optional fourth ingredient, the mixture to be provided according to step a) of the process of the present invention may optionally comprise one or more excipients. Suitable excipients as referred to herein are known to the skilled person. For example, the mixture to be provided according to step a) of the present invention may optionally contain pH-correcting agents in order to adjust the pH, such as physiologically acceptable bases, acids or salts, optionally as buffer mixtures. In this context, the term “physiologically acceptable” does not mean that one of the excipients must be tolerable on its own and in undiluted form, which would not be the case, for example, for sodium hydroxide solution, but means that it must be tolerable at the concentration in which it is contained in the lyophilized pharmaceutical composition, especially after reconstitution.

Suitable pH-correcting agents or buffers for adjusting the pH may be selected, inter alia, with regard to the intended route of application. Examples for potentially useful excipients of this group comprise sodium hydroxide solution, basic salts of sodium, calcium or magnesium such as, for example, citrates, phosphates, acetates, tartrates, lactates etc., amino acids, acidic salts such as hydrogen phosphates or dihydrogen phosphates, especially those of sodium, moreover, organic and inorganic acids such as, for example, hydrochloric acid, sulphuric acid, phosphoric acid, citric acid, cromoglycinic acid, acetic acid, lactic acid, tartaric acid, succinic acid, fumaric acid, lysine, methionine, acidic hydrogen phosphates of sodium or potassium etc.

In further specific embodiments, the mixture to be provided according to process step a) of the present invention may comprise one or more further excipients which are selected from chelating agents, for example, disodium edetate dihydrate, calcium sodium EDTA, preferably disodium edetate dihydrate.

Furthermore, the mixture to be provided according to step a) of the present invention may or may not contain as an excipient osmotically active adjuvants in order to adjust it to a desired osmolality after reconstitution, which is important in certain applications such as especially for inhalation, in order to achieve good tolerability. Such adjuvants are frequently referred to as “isotonizing agents” even if their addition does not necessarily result in an isotonic composition after reconstitution, but in an isotonicity close to physiological osmolality in order to achieve the best possible physiological tolerability.

A particularly frequently used isotonizing agent is sodium chloride, but this is not suitable in every case. In an advantageous embodiment of the invention, the mixture according to process step a) contains no sodium chloride, except, of course, natural ubiquitous sodium chloride amounts which may also be contained in water of pharmaceutical quality. In another embodiment, the mixture according to process step a) contains an essentially neutral salt as isotonizing agent which is not sodium chloride, but, for example, a sodium sulphate or sodium phosphate. It should be noted, however, that the isotonizing agent may also be comprised by the aqueous carrier liquid, for example in form of an aqueous solution of sodium chloride (saline). In this case, however, salts other than sodium salts may be also preferable. Thus, it is known that certain calcium and magnesium salts have a positive or supporting effect in the inhalation of active agent solutions, possibly because they themselves counteract the local irritations caused by the administration and because they have a bronchodilatory effect which is currently postulated in the clinical literature (for example Hughes et al., Lancet. 2003; 361 (9375): 2114-7) and/or because they inhibit the adhesion of germs to the proteoglycans of the mucosa of the respiratory tract so that the mucociliary clearance as the organism's natural defense against pathogens is supported indirectly (K. W. Tsang et al., Eur. Resp. 2003. 21, 932-938). Advantageous may be, for example, magnesium sulphate, which has excellent pulmonary tolerability and can be inhaled without concern, as well as calcium chloride (1-10 mmol).

In further specific embodiments, suitable excipients that may be added to the mixture to be provided according to step a) of the present invention comprise saccharides or sugars, such as disaccharides as described in further detail below.

As a further ingredient, the mixture to be provided according to step a) of the present invention comprises an aqueous carrier liquid or aqueous liquid vehicle. The aqueous carrier liquid or vehicle may be water or an aqueous solution of pharmaceutically acceptable salts or isotonizing agents and preferably may be sterile. In other preferred embodiments, however, the sterile aqueous carrier liquid is water, preferably sterilized or sterile water, such as water that is suitable for injections.

In specific embodiments of the process of the present invention, the amount of the membrane-forming substance selected from the group of phospholipids, preferably the lecithin to be provided in the mixture according to step a) is larger than the amount of the solubility-enhancing substance selected from the group of non-ionic surfactants. In exemplary embodiments, the weight ratio of the membrane forming substance selected from the group of phospholipids, preferably the lecithin, to the solubility enhancing substance selected from the group of non-ionic surfactants, preferably the polysorbate, is selected in the range of from about 15:1 to about 9:1, preferably from about 14:1 to about 12:1, for example, about 13:1.

In further specific embodiments, the weight ratio between the (sum of the) membrane-forming substance(s) selected from the group of phospholipids and the solubility-enhancing substance selected from the group of non-ionic surfactant on the one hand and inhalable macrocyclic active ingredient, specifically CsA, to be provided in the mixture according to step a) on the other hand is selected in the range of from about 5:1 to about 20:1, preferably from about 8:1 to about 12:1 and more preferably about 9:1.

In yet further specific embodiments, the weight ratio between the membrane-forming substance selected from the group of phospholipids, preferably the lecithin, the solubility-enhancing substance selected from the group of non-ionic surfactants, preferably the polysorbate and the inhalable immunosuppressive macrocyclic active ingredient, specifically CsA, is selected in the range of from about 15:1:1.5 to about 5:0.3:0.5, and preferably at about 9:0.7:1.

In further specific embodiments, the weight ratio of the membrane forming substance selected from the group of phospholipids as described above to the inhalable immunosuppressive macrocyclic active ingredient, specifically CsA, is selected in the range of from about 8:1 to about 11:1, preferably from about 8.5:1 to about 10:1, for example, about 9:1.

In exemplary embodiments, the mixture to be provided according to process step a) of the present invention may comprise the inhalable immunosuppressive macrocyclic active ingredient, specifically CsA, in a concentration selected within the range of from about 1 g/L to about 7 g/L, or from about 2 g/L to about 6 g/L or from about 3 to about 5 g/L, for example 4 g/L.

In further exemplary embodiments, the mixture to be provided according to process step a) of the present invention may comprise the membrane-forming substance selected from the group of phospholipids as described above, specifically the Lipoid such as Lipoid S100, in a concentration selected within the range of from about 20 g/L to about 60 g/L, or from about 30 g/L to about 50 g/L, or from about 30 g/L to about 40 g/L.

In yet further exemplary embodiments, the mixture to be provided according to process step a) of the present invention may comprise the solubility-enhancing substance selected from the group of non-ionic surfactants as described above, specifically the polysorbate such as polysorbate 80, in a concentration selected within the range of from about 1 g/L to about 5 g/L, specifically selected within the range of from about 2 g/L to about 4 g/L, or from about 2.5 g/L to about 3.5 g/L.

In specific embodiments, the mixture to be provided according to process step a) of the present invention may comprise at least one saccharide or sugar, specifically at least one disaccharide as an excipient. The disaccharide that may be comprised by the mixture according to process step a) may, in specific embodiments, be selected from the group consisting of saccharose (sucrose; the terms ‘saccharose’ and ‘sucrose’ as used herein have the same meaning and are used synonymously for β-D-Fructofuranosyl α-D-glucopyranoside; CAS number 57-50-1), lactose (β-D-Galactopyranosyl-(1→4)-D-glucose; CAS number 63-42-3) and trehalose (α-D-glucopyranosyl-(1→1)-α-D-glucopyranoside; CAS number 99-20-7). In specific embodiments the mixture provided according to process step a) comprises saccharose as an excipient.

In further specific embodiments of the mixture to be provided according to process step a) of the present invention, the ratio of the weight of the chosen at least one saccharide or disaccharide, preferably sucrose, to the weight of cyclosporine A in the mixture to be provided according to step a) of the process of the present invention is selected in the range of from about 10:1 to about 30:1, or from about 20:1 to about 30:1 or from about 20:1 to about 27.5:1 or from about 22.5:1 to about 27.5:1.

Accordingly, in further exemplary embodiments, the mixture to be provided according to step a) of the present invention may comprise a disaccharide, specifically saccharose, trehalose and/or lactose, especially saccharose in a concentration selected within the range of from about 60 g/L to about 140 g/L, or from about 80 g/L to about 120 g/L, or from about 90 g/L to about 110 g/L.

For providing the mixture according to step a) of the process of the present invention, the chosen ingredients as described above may be added to a suitable vessel, such as the steering vessel, and may be stirred using standard techniques until a homogeneous mixture results. In specific embodiments, the ingredients may be added to the vessel together at once or consecutively, as appropriate. For example, the chosen aqueous carrier liquid or vehicle such as sterilized water may be added to the vessel as the first ingredient.

In further exemplary embodiments, the chosen excipients may then be added to the aqueous carrier liquid, specifically water, and steered until sufficient or complete dissolution is reached. To the resulting mixture or solution the further ingredients may be added altogether at once or consecutively. For example, the chosen membrane-forming substance selected from the group of phospholipids may be added and the resulting mixture steered until a sufficiently homogeneous dispersion results. Following that, the chosen solubility enhancing substance selected from the group of non-ionic surfactants may be added to the mixture and steered until a sufficiently homogeneous dispersion is formed. Finally, according to exemplary embodiments, the chosen inhalable immunosuppressive macrocyclic active ingredient, specifically CsA, may be added and steered until a sufficiently homogeneous dispersion is formed.

The preparation of the mixture according to process step a) of the present invention may be conducted at about room temperature, or, depending on the specific components to be added or dissolved, above or below room temperature, usually at a temperature selected within the range of from about 0° C. or from about 5° C. to about 45° C. or to about 50° C. In specific embodiments, some components, specifically excipients or salts, may be added at elevated temperature, for example in the range of from about 35° C. to about 50° C. or from about 40° C. to about 45° C. and following that the temperature may be lowered, e.g. to room temperature or below, such as to a temperature selected within the range of from about 0° C. or from about 5° C. to about 25° C. or from about 15° C. to about 25° C., depending on the further ingredients or components to be added or dissolved. Optionally, for example to complete dissolution of specific ingredients, further amounts of water or the chosen aqueous carrier liquid may be added to the mixture or may be removed from the mixture by techniques know to the skilled person. The resulting liquid aqueous mixture may then be subjected to process step b) as described below.

According to step b) of the process of the present invention, the mixture as provided in step a) is dispersed to form an intermediate aqueous dispersion comprising the at least one inhalable immunosuppressive macrocyclic active ingredient, specifically cyclosporine A, in the aqueous carrier liquid. For performing the dispersion according to step b), the mixture as prepared according to step a) as described above may be transferred to a vessel suitable for dispersing the mixture or, in alternative embodiments, the dispersing may be performed in the same vessel of apparatus as used in step a). Suitable vessels may be, for example, vessels made of a material suitable for the preparation of pharmaceutical compositions such as suitable polymeric materials or preferably stainless steel. Depending on the overall final volume of the mixture to be dispersed, the vessel may have a volume usually in the range of from about 100 L to about 1,000 L, often from about 400 L to about 700 L. In specific embodiments, the volume of the vessel is chosen to substantially exceed the final overall volume of the mixture to dispersed, in specific embodiments by at least 50% or even by at least 10% of the overall final volume of the mixture to be dispersed. In further specific exemplary embodiments, the vessel may have an (inner) diameter within the range of from about 700 mm to about 1,400 mm of from about 700 mm to about 1,000 mm, or from about 850 mm to about 950 mm or to about 920 mm.

In general, the dispersing according to step b) of the process of the present invention is conducted using a disperser suitable for dispersing liquid mixtures, specifically aqueous liquid mixtures comprising further constituents as described above in amounts and concentrations as also described in detail above. The chosen disperser may, for example, be mobile, or in other words may be immersed and removed from the aqueous mixture to be dispersed. In further embodiments, however, the disperser may be firmly attached or integrated into the vessel in which the dispersing is conducted. In further embodiments, however, the disperser may be placed between two vessels as an inline disperser as described in further detail below.

In specific embodiments, the dispersing according to step b) is conducted using a rotor-stator-type disperser. Rotor-stator-type dispersers are known to those of skill in the art and are commercially available, for example from IKA-Werke GmbH & Co KG, Germany, such as ULTRA-TURRAX® UTE batch dispersers. Rotor-stator-type dispersers usually comprise a rotor adapted to be rotated at high speed in a corresponding stator, whereas both, the rotor as well as the stator are immersed in the mixture to be dispersed. In specific embodiments, the rotor and/or the stator comprise a multiplicity of teeth. Usually, these teeth are firmly attached to a base plate of the rotor or the stator and are preferably oriented parallel to the main axis of rotation of said rotor or of the main drive shaft of the disperser connecting the motor of the disperser with the rotor. The teeth may be arranged around the corresponding circumference of the rotor and/or the stator, respectively, usually in the form of rows of teeth. It should be noted that both, the rotor and/or the stator may have just one row of teeth or a multiplicity of rows of teeth which are then usually arranged in a concentric manner with regard to the main axis of rotation of the disperser. The mixture to be dispersed, due to the rotation of the rotor, usually is forced through the gaps between the teeth or rows of teeth of the rotor and/or the stator whereby a shear force is exerted on the mixture to be dispersed.

In further specific embodiments, the dispersing according to step b) of the present invention may also be conducted using an inline disperser. Inline dispersers are commercially available e.g. from IKA-Werke GmbH & Co KG, Germany, such as ULTRA-TURRAX® UTL inline dispersers. Such inline dispersers allow, for example, for the continuous dispersing of the mixture or dispersing from a first vessel A to a second vessel B and vice versa whereby the mixture to be dispersed is continuously charged to an (external) disperser.

As a measure for the sheer forces exerted on the mixture to be dispersed, the “shear rate” or, in other words, “shear velocity” may be used. The term “shear rate” as used herein, especially when used in connection with a rotor-stator-type disperser, having the dimension of [1/s] or [s⁻¹], may be determined by dividing the circumferential speed of the rotor as measured in [m/s], as described in further detail below, by the distance or in other words by the width of the gap between the rotor and the stator as measured in [m] according to Formula (I):

F_{R}=vᵤ/ds (I)

wherein F_{R} means the shear rate, vᵤ means the circumferential speed of the rotor and ds means the width of the gap between stator and rotor and wherein the circumferential speed of the rotor is calculated according to Formula (II):

vᵤ=d·π·n/60 (II)

wherein d is the diameter of the rotor and n denotes the rotational speed of the rotor in rpm (revolutions per minute).

In specific embodiments in which a disperser of the rotor-stator-type as described above is used, the rotor may generally have diameter within the range of from about 50 mm to about 150 mm or from about 60 to about 140 mm. Specifically in cases in which an immersion or batch disperser as described above is used, the rotor may have a diameter within the range of from about 80 mm to about 120 mm or within the range of from about 90 mm to about 110 mm, such as from about 95 mm to about 105 mm. In further specific embodiments in which an inline disperser as described in further detail below is used, the rotor may have diameter selected within the range of from about 100 mm to about 140 mm or from about 110 to about 130 mm. In further embodiments, the corresponding stator usually is adapted to surround the corresponding rotor. Accordingly, the (inner) diameter of the space in which the corresponding rotor is received usually corresponds to the diameter of the rotor plus two times the distance of the gap between the rotor and the stator. This gap, in specific embodiments, usually does not exceed about 4 mm or about 3 mm or about 2 mm and may, especially in cases in which an immersion or batch disperser is used, be selected within the range of from about 0.5 to about 2 mm, or from about 0.5 to about 1.5 mm, or from about 0.7 to about 0.9 mm. In further specific embodiments, especially in cases in which an inline disperser is used, the gap (shear gap) may be selected within a range of from about 0.1 mm to about 0.6 mm, or from about 0.2 mm to about 0.5 mm, or from about 0.3 to about 0.4 mm.

In specific embodiments, the dispersing according to step b) is performed at a shear rate (shear velocity) of at least 22,000 1/s (twenty-two thousand 1/s), or at least 24,000 1/s, or at least 25,000 1/s. In further specific embodiments, the dispersing according to step b) may be performed at a shear rate (shear velocity) selected within the range of from about 22,000 1/s to about 120,000 1/s, or from about 22,000 1/s to about 100,000 1/s. In further specific embodiments, especially in cases in which an immersion or batch disperser is used, the dispersing according to process step b) may be performed at a shear rate of from about 24,000 1/s to about 80,000 1/s or to about 60,000 1/s or from about 25,000 1/s to about 55,000 1/s or from about 22,000 1/s or from about 25,000 1/s to about 40,000 1/s or from about 25,000 1/s to about 38,000 1/s or from about 27,000 1/s or from about 28,000 1/s to about 37,000 1/s or from about 30,000 to about 37,000 1/s.

In further specific embodiments, especially in cases in which an inline disperser is used, the dispersing according to process step b) may be performed at a shear rate of from about 40,000 1/s to about 110,000 1/s or to about 100,000 1/s or from about 45,000 1/s to about 90,000 1/s or from about 50,000 1/s or from about 55,000 1/s to about 85,000 1/s or from about 60,000 1/s to about 85,000 1/s or from about 65,000 1/s to about 75,000 1/s.

The rotor and the stator, in specific embodiments, each may have a plurality of teeth as described above. The number of the teeth connected to the stator and to the rotor may be chosen independently from each other. In specific embodiments, the number of teeth of the rotor and the stator each is at least about 10, or at least about 15, or at least about 20. The number of teeth of the rotor, in general may be chosen within a broad range often exceeding 50, 75 or even 100 teeth each. In specific embodiments, especially in cases in which an immersion or batch disperser is used, the rotor may have a number of teeth selected within the range of from about 10 to about 75, or from about 10 or 15 to about 40 or 50, or from about 20 to about 35. The number of teeth of the stator, in general may be independently chosen within a broad range often exceeding 50, 75 or even 100 teeth each. In specific embodiments, especially in cases in which an immersion or batch disperser is used, the stator has a number of teeth selected within the range of from about 10 to about 75, or from about 10 or 15 to about 40 or 50, or from about 20 to about 35. In these cases, the individual teeth may be spaced apart from each other in usually by about 15 or 10 mm or below, such as selected within a range of from about 1 mm to about 8 mm or from about 2 mm to about 6 mm such as about 4 mm.

In further specific embodiments, especially in case in which an inline-disperser is used, the rotor may have a number of teeth selected within the range of from about 10 to about 75, or from about 20 or 30 to about 60 or 50, or from about 35 to about 45. In these cases, the stator may have a number of teeth selected within the range of from about 30 to about 80, or from about 35 or 40 to about 75 or 70, or from about 45 or 40 to about 60. In these cases, the individual teeth may be spaced apart from each other in usually by about 10 or 5 mm or below, such as selected within a range of from about 0.5 mm to about 5 mm or from about 1 mm to about 2.5 mm. Especially in cases in which an inline disperser is used, the distance between the teeth of the stator relative to each other and the teeth of the rotor relative to each other may differ, such as from about 1.0 mm to about 2.0 mm, for example 1.6 mm for the stator and from about 1.5 mm to about 2.5 mm, for example 2.0 mm for the rotor.

As a further measure for the shear forces exerted on the mixture to be dispersed according to step b) of the process of the present invention, the “shear frequency” may be used. The term “shear frequency” as used herein, especially when used in connection with a rotor-stator-type disperser, having the dimension of [1/s] or [s⁻¹], may be determined by multiplying the rotational speed of the rotor [1/s] with the number of teeth of the rotor and the number of teeth of the stator according to Formula (III):

Fₛ=n·S_{Rotor}·S_{Stator} (III)

wherein Fₛ denotes the shear frequency, n is the rotational speed of the rotor as measured in [1/s] and S_{Rotor} and S_{Stator} mean the number of teeth of the rotor and the stator, respectively.

In specific embodiments, the dispersing according to step b) of the process of the present invention is performed at a shear frequency of at least 42,000 1/s, or at least 44,000 1/s, or at least 45,000 1/s. In further specific embodiments, especially in cases in which an immersion or batch disperser is used, the dispersing according to step b) is performed at a shear frequency selected within the range of from about 42,000 1/s to about 140,000 1/s, or from about 42,000 1/s to about 120,000 1/s or from about 45,000 1/s to about 100,000 1/s. In yet further specific embodiments, in these cases the shear rate may be selected within the range of from about 50,000 1/s to about 140,000 or to about 100,000 1/s or from about 50,000 1/s to about 80,000 1/s or from about 55,000 1/s to about 75,000 1/s or to about 70,000 1/s.

In yet further specific embodiments, the dispersing according to step b) of the process of the present invention, especially in cases in which an immersion or batch disperser is used, may be performed at a combination of a specific shear rate with a specific shear frequency, such as at a shear rate (shear velocity) of at least 22,000 1/s (twenty-two thousand 1/s), or at least 24,000 1/s, or at least 25,000 1/s and at a shear frequency of at least 42,000 1/s, or at least 44,000 1/s, or at least 45,000 1/s. In further specific embodiments in which an immersion disperser is used the dispersion according to step b) may be performed a shear rate selected within the range of from about 28,000 1/s to about 37,000 1/s or from about 30,000 to about 37,000 1/s and a shear frequency of from about 50,000 1/s to about 80,000 1/s or from about 55,000 1/s to about 75,000 1/s or to about 70,000 1/s.

In further specific embodiments, especially in cases in which an inline-disperser as described above is used, the dispersing according to step b) may be performed at a shear frequency selected within the range of from about 100,000 1/s to about 200,000 1/s, or from about 110,000 1/s to about 190,000 1/s or from about 120,000 1/s to about 180,000 1/s. In yet further specific embodiments, in these cases the shear rate may be selected within the range of from about 130,000 1/s to about 170,000 or from about 140,000 1/s to about 160,000 1/s.

In yet further specific embodiments in which an inline disperser as described above is used, the dispersing according to step b) may be performed at a shear rate selected in the range of from about 55,000 1/s to about 85,000 1/s or from about 60,000 1/s to about 85,000 1/s or from about 65,000 1/s to about 75,000 1/s and a shear frequency selected within the range of from about 120,000 1/s to about 180,000 1/s or from about 130,000 1/s to about 170,000 or from about 140,000 1/s to about 160,000 1/s.

As already mentioned above, the dispersing according to step b) may be conducted using an immersion disperser, or in other words a disperser that is fixedly arranged in the dispersing vessel or which can be removeable immersed into the mixture to be dispersed. In alternative embodiments, however, the dispersing according to step b) may be conducted using an inline disperser as described above. In further embodiments, however, the dispersing according to step b) of the present invention may be conducted using both an immersion disperser (or a plurality of immersion dispersers simultaneously) and an inline disperser. In further specific embodiments, the immersion disperser and the inline disperser may be used consecutively. For example, in specific embodiments the dispersing according to step b) of the present invention may be conducted using an immersion disperser first, specifically under conditions as described in detail above, followed by dispersing using an inline disperser under conditions as also described in detail above. In further specific embodiments, the conditions chosen for the (partial) dispersion conducted with an immersion disperser, especially with regard to the chosen shear rate, shear frequency and/rotational speed of the rotor differ from the (partial) dispersion conducted with an inline disperser.

As discussed above, the rotor of the rotor stator-type disperser that may be used in step b) of the process of the present invention may have a multiplicity of teeth which may be arranged along a perimeter or circumferential perimeter of the rotor and in which the multiplicity of teeth preferable have the same distance to the respective neighbouring teeth in the row. Furthermore, the rotor may or may not have a multiplicity of rows of teeth which are usually arranged in a concentric manner with regard to the main rotational axis of the rotor. Accordingly, in specific embodiments, especially in cases in which an immersion disperser is used, the rotor may have multiple rows of teeth, such as 2 to 4 rows of teeth, or 2 or 3 rows of teeth.

Furthermore, the corresponding stator also may or may not have a multiplicity of rows of teeth which are usually arranged in a concentric manner with regard to the main rotational axis of the stator. Accordingly, in specific embodiments, the stator has multiple rows of teeth, such as 2 to 4 rows of teeth, or 2 or 3 rows of teeth. In further specific embodiments, rotor and stator combined have a total of 2 to 8, specifically 3 to 6 rows of teeth. In further specific embodiments, especially in cases in which an inline disperser is used, the rotor and stator may also have multiple rows of teeth, such as 3 to 12 rows of teeth combined, or 6 to 10 rows of teeth combined, for example 8 rows of teeth for rotor and stator combined.

The dispersing according to step b) of the process of the present invention to provide the intermediate aqueous dispersion comprising the inhalable immunosuppressive macrocyclic active ingredient in the aqueous carrier liquid is usually conducted at high rotational speed of the rotor of the disperser, usually at a rotational speed of up to about 10,000 rpm or up to about 8,000 rpm (revolutions per minute). In specific embodiments, especially in cases in which an immersion disperser is used, the dispersing may be conducted at a rotational speed (of the rotor of the disperser) selected within the range of from about 2,000 rpm or from about 3,000 rpm to about 6,000 rpm or of from about 3,000 rpm or about 4,000 rpm to about 5,500 rpm. Depending on the diameter of the chosen rotor, this results in a circumferential speed of the rotor of at least about 10 m/s. In specific embodiments, the dispersing according to step b) is conducted with a circumferential speed of the rotor selected within the range of from about 15 m/s to about 40 m/s, or from about 15 m/s to about 30 m/s.

In further specific embodiments, especially in cases in which an inline disperser is used, the dispersing may be conducted at a rotational speed (of the rotor of the disperser) selected within the range of from about 2,000 rpm or from about 3,000 rpm to about 6,000 rpm or of from about 3,000 rpm or about 3,500 rpm to about 4,500 rpm.

In order to achieve such circumferential speeds, in specific embodiments, the dispersing according to step b) is conducted using a disperser with a motor having a power of at least 2 kW, specifically with a motor having a power selected within the range of from about 2 kW to about 10 kW, or from about 3 to about 8 kW.

In order to prepare the intermediate aqueous dispersion comprising the inhalable immunosuppressive macrocyclic active ingredient in the aqueous carrier liquid the dispersing according to step b) is performed for a period of time, usually exceeding 10 min or 15 min or 1 h or even 2 h. In many cases, especially when an immersion disperser or an inline disperser alone is used, the dispersing according to step b) is performed for a (total) period of at least about 1 h or at least about 3 h, for example for a period selected within the range of from about 1 h to about 8 h, or from about 3 h to about 8 h, or from about 3 h to about 5 h or from about 1 h to about 4 h.

In further specific embodiments, as described in detail above, the dispersing according to step b) of the process of the present invention may be conducted using an immersion-disperser as well as an inline disperser, wherein the immersion disperser and the inline disperser are used consecutively. In specific embodiments, the dispersing may be started using an immersion-disperser (or a plurality of immersion dispersers) followed by an inline disperser. In these cases, the dispersing times can also be reduced. For example, dispersing using an immersion disperser may be conducted for a period of up to about 1 h or up to about 30 min, for example from about 10 to about 30 min, followed by dispersing using an inline disperser for a period of up to 6 h or up to 4 h, for example for a period of from about 1 h to about 4 h, which may help to effectively reduce the overall duration of the dispersing step.

In further specific embodiments, the dispersing according to step b) may be performed at a temperature (of the mixture to be dispersed) selected within the range of from about 15° C. to about 35° C., or from about 15° C. to about 30° C., or from about 15° C. to about 25° C. or from about 20° C. to about 25° C. In order to avoid a temperature raise, the mixture may be cooled using standard techniques, if necessary at all. In further specific embodiments, the dispersing according to step b) is performed at ambient (atmospheric) pressure.

The resulting intermediate aqueous dispersion comprising the at least one inhalable immunosuppressive macrocyclic active ingredient, specifically CsA, in the aqueous carrier liquid obtained according to process step b) already comprises the inhalable immunosuppressive macrocyclic active ingredient in liposomally solubilized form, specifically liposomally solubilized CsA (L-CsA) to a certain extent.

However, to complete the solubilization of the chosen inhalable immunosuppressive macrocyclic active ingredient, specifically CsA, and to further reduce the particle size of the formed liposomes, the resulting mixture is subjected to further homogenization according to the following process step c) as described below.

According to step c) of the process of the present invention, the intermediate aqueous dispersion as formed in step b) is homogenized to form the dispersion comprising the inhalable immunosuppressive macrocyclic active ingredient in liposomally solubilized form, wherein, preferably, at least about 90% to about 100% of the inhalable immunosuppressive active ingredient, specifically CsA, of the total amount of said compound as introduced according to step a) is present in liposomally solubilized form.

According to process step c), the resulting intermediate aqueous dispersion is then exposed to homogenization conditions to generate a colloidal dispersion of the inhalable immunosuppressive macrocyclic active ingredient, specifically cyclosporine A, in liposomally solubilized form. In preferred embodiments, the step of homogenizing the intermediate aqueous dispersion formed in step b) to form the dispersion comprising the inhalable immunosuppressive macrocyclic active ingredient in liposomally solubilized form comprises high pressure homogenisation, as known to those of skill in the art. In exemplary embodiments, the high-pressure homogenisation according to step c) may be conducted using a piston-gap-type homogeniser which may comprise one or more, such as one to six plungers or two to four plungers, specifically three plungers. In further embodiments, such piston-gap-type homogenizer may comprise a homogenization valve, specifically a ceramic homogenization valve such as a NanoVALVE (available from GEA, Italy). In further specific embodiments, the homogenization pressure may be applied in a 2-step pressure cascade as described in further detail below. Exemplary homogenizers suitable for conducting the high-pressure homogenization according to step c) comprise, but are not limited to, high-pressure homoMicrofluidics M-110EH or Ariete NS3006L (GEA, Italy).

In further embodiments, the high-pressure homogenization according to step c) may be carried out a single time or several times repeatedly. Specifically, the high-pressure homogenization may be carried out repeatedly, such as about 5 to about 15 times. Furthermore, the high-pressure homogenization may be carried out at any suitable pressure, usually at pressures of up to about 1,500 bar, or at pressures in the range of from about 50 to about 1,500 bar or at pressures selected within the range of from about 100 to about 1,000 bar. Preferably, high-pressure homogenization may be carried out repeatedly, such as about 5 to about 15 times at pressures in the range of from about 100 to about 1,000 bar, if necessary, under reduced pressure.

In further embodiments, the homogenization according to step c) of the process of the present invention may be conducted in a 2-step pressure cascade in which a relatively lower pressure is applied in a first stage and a relatively higher pressure is applied in a second stage. Exemplary pressure ranges for the first stage may be selected within the pressure ranges as described in general above, such as within a range of from about 50 to about 200 bar, specifically from about 75 to about 125 bar, such as about 100 bar. Exemplary pressure ranges for the second stage may be selected within the pressure ranges as described in general above, such as within a range of from about 500 to about 1,500 bar, specifically from about 750 to about 1,250 bar, such as about 1,000 bar. As described above, when conducted in a 2-step pressure cascade, the homogenization step may be conducted repeatedly, such as about 5 to about 15 times. In further embodiments, the high-pressure homogenization according to step c) may be conducted at temperatures of up to about 35° C. to 37° C. (temperature of the dispersion to be homogenized), specifically at a temperature within the range of from about 2° C. to about 35° C., or from about 2° C. to about 25° C., or from about 5° C. or 7° C. to about 25° C. In specific embodiments, the homogenization of the intermediate aqueous dispersion as received in step b) of the process of the present invention or at least a part thereof may be conducted at temperature below room temperature, for example at temperatures in the range of from about 2° C. to about 10° C. In these cases, but also in cases in which the homogenization is conducted at higher temperatures as described above a heat exchanger may be used to cool the aqueous dispersion during homogenization.

After completion of the homogenization according to process step c) of the present invention a homogenized dispersion is obtained comprising a colloidal dispersion of the inhalable immunosuppressive macrocyclic active ingredient, specifically cyclosporine A, in liposomally solubilized form consisting of mainly unilamellar liposomes having a mean diameter of at most 100 nm, such as from about 30 nm to about 70 nm, and/or a polydispersity index (PI) of not more than 0.50, or even not more than 0.40, such from about 0.15 to about 0.2, and preferably appears as a clear opalescent solution without visible particles.

After completion of the homogenization according to step c), according to an optional step c1) the resulting homogenized dispersion comprising the inhalable immunosuppressive macrocyclic active ingredient, specifically cyclosporine A, in liposomally solubilized form may, if necessary, be sterilized, for example by sterile filtration. Suitable filters for such filtration to remove or reduce potential bioburden or microbial contaminants include, but are not limited to, for example, Fluorodyne® EX (PALL) with a pore size of 0.2 μm, and others.

In further specific embodiments it may be useful to filtrate the intermediate aqueous dispersion comprising the inhalable immunosuppressive macrocyclic active ingredient in the aqueous carrier liquid as formed in step b) prior to homogenizing according to step c). Accordingly, the process according to this first aspect of the present invention may comprise as a further step
b1) filtrating of the intermediate aqueous dispersion comprising the inhalable immunosuppressive macrocyclic active ingredient in the aqueous carrier liquid as formed in step b) prior to homogenizing the resulting filtered intermediate aqueous dispersion according to step c).

According to these embodiments, the intermediate aqueous dispersion comprising the inhalable immunosuppressive macrocyclic active ingredient, specifically cyclosporine A, in the aqueous carrier liquid as formed in step b) is filtered before it is further processed by high-pressure homogenization as described above in connection with process step c). This optional additional filtration might be helpful to avoid mechanical stress and potential damage of the high-pressure homogenizer due to potential residual particles in the intermediate aqueous dispersion comprising the inhalable immunosuppressive macrocyclic active ingredient, specifically cyclosporine A. The filtration may be performed using readily available filters or filter materials that are suitable for the contact with pharmaceutical compounds or compositions, such as steel or a suitable polymeric material. In specific embodiments, the filtration according to this optional step b1) is performed using a filter with a mean pore width within the range of from about 75 μm or from about 100 μm to about 300 μm or from about 150 μm to about 250 μm or from about 200 μm to about 250 μm or from about 220 μm to about 250 μm or to about 230 μm, such as about 225 μm.

The process according to this first aspect of the inventions provides a dispersion comprising the inhalable immunosuppressive macrocyclic active ingredient, specifically cyclosporine A in liposomally solubilized form as the product of process step c) as described above. The inhalable immunosuppressive macrocyclic active ingredient, specifically cyclosporine A, preferably in a therapeutically effective amount, may be comprised by the liposome-forming structures formed by the membrane-forming substance selected from the group of phospholipids and the solubility-enhancing substance selected from the group of non-ionic surfactants as outlined above.

In specific embodiments, the inhalable immunosuppressive macrocyclic active ingredient, specifically cyclosporine A, is at least partially incorporated (or intercalated) in the bilayer membrane of the liposome-forming structures. The term “incorporated” as used herein means, with regard to specifically CsA being a lipophilic compound, that CsA is located or intercalated in the inner lipophilic part of the bilayer lipid membrane rather than on the hydrophilic outer surfaces of the lipid bilayer membrane (whereas the term “surfaces” can mean both surfaces, or more specifically the inner or outer surface of the bilayer membrane forming the liposome-forming structures).

In preferred embodiments, the inhalable immunosuppressive macrocyclic active ingredient, specifically cyclosporine A is predominantly incorporated (or intercalated) in the bilayer membrane of the liposome-forming structures. In exemplary embodiments, at least about 90% or even at least about 95% or even at least about 97.5% of the total amount of the inhalable immunosuppressive macrocyclic active ingredient, specifically CsA, is comprised in the bilayer membranes of the liposome-forming structures as formed according to the process of the present invention. In further exemplary embodiments, at least about 90% or about 95% to about 97.5% or to about 99% or 99.5% or even 99.9% of the total amount of the inhalable immunosuppressive macrocyclic active ingredient, specifically CsA is incorporated in the bilayer membranes of the liposome-forming structures formed in the process of the present invention.

In a second aspect, the present invention provides a process for the preparation of a lyophilized pharmaceutical composition for reconstitution in an aqueous carrier liquid, the lyophilized pharmaceutical composition comprising an inhalable immunosuppressive macrocyclic active ingredient in liposomally solubilized form,

wherein the process comprises the preparation of a dispersion comprising an inhalable immunosuppressive macrocyclic active ingredient in liposomally solubilized form in an aqueous carrier liquid according to the process of the first aspect of the invention as described above; and further comprising the step of
d) removing the aqueous carrier liquid at least partially under lyophilization conditions to form the lyophilized pharmaceutical composition.

In other words, the present invention also provides a process for the preparation of a lyophilized pharmaceutical composition for reconstitution in an aqueous carrier liquid, the lyophilized pharmaceutical composition comprising an inhalable immunosuppressive macrocyclic active ingredient, specifically cyclosporine A, in liposomally solubilized form, the process comprising the steps of
a) providing a mixture comprising
   the inhalable immunosuppressive macrocyclic active ingredient, specifically CsA;
   a membrane-forming substance selected from the group of phospholipids;
   a solubility-enhancing substance selected from the group of non-ionic surfactants;
   optionally one or more excipients; and
   the aqueous carrier liquid;
b) dispersing the mixture as provided in step a) to form an intermediate aqueous dispersion comprising the inhalable immunosuppressive macrocyclic active ingredient in the aqueous carrier liquid;
b1) optionally filtrating of the intermediate aqueous dispersion comprising the inhalable immunosuppressive macrocyclic active ingredient in the aqueous carrier liquid as formed in step b);
c) homogenizing the intermediate aqueous dispersion as formed in step b) or b1) to form the dispersion comprising the inhalable immunosuppressive macrocyclic active ingredient in liposomally solubilized form;
c1) optionally sterilizing the dispersion comprising the inhalable immunosuppressive macrocyclic active ingredient in liposomally solubilized form; and
d) removing the aqueous carrier liquid at least partially under lyophilization conditions to form the lyophilized pharmaceutical composition.

It should be understood that all features, embodiments, starting materials, process conditions and combinations thereof as described above in connection with the process of the first aspect of the invention also apply to the process according to this second aspect of the invention.

In specific embodiments of this second aspect of the invention also, the dispersing according to process step b) may be performed using an immersion disperser and an inline disperser as described above, wherein preferably the immersion disperser and the inline disperser are used consecutively.

The process of this second aspect of the invention comprises, in addition to the process steps of the process of the first aspect of the invention an additional step d) in which the aqueous carrier liquid is at least partially removed under lyophilization conditions to form a lyophilized pharmaceutical composition.

The lyophilization according to process step d) can be conducted according to standard techniques known to those of skill in the art, for example by using a LyoStar MNL-055-A/LSACC3E lyophilizer or a GEA Lyovac® GT 400-D. The lyophilization to form the lyophilized pharmaceutical compositions of this aspect of the invention may be conducted in continuous manner, for example at constant pressure and temperature or preferably may be conducted stepwise, wherein each step of the lyophilization protocol or process may be conducted at specific pressures, temperatures and for a defined duration. In exemplary embodiments, the lyophilization process or cycle may comprise up to 20, or from about 2 to about 15, preferably from about 5 to about 15 consecutive steps. Each step may, for example, be conducted at temperature within the range of from about 40° C. to about −60° C., preferably from about 20° C. to about −50° C., either at a constant temperature or at temperatures that may be raised or lowered at a certain gradient. Furthermore, each lyophilization step may be conducted at reduced pressures, for example at pressures below ambient pressure, such as in the range from about 0.005 mbar to about 800 mbar, preferably from about 0.009 mbar to about 0.500 mbar, or to about 0.400 mbar or to about 0.300 mbar.

It should be noted that, in addition to the lyophilization as described above, some of the aqueous carrier liquid to be removed according to process step d) may also be removed by other techniques known to those of skill in the art, for example by distillation under reduced pressure, especially prior to lyophilization. Furthermore, it may be advantageous to prepare aliquots of the dispersion comprising the inhalable immunosuppressive macrocyclic active ingredient in liposomally solubilized form as received in process step c) of smaller volume containing a determined amount of the active ingredient.

In this context it should be noted that while the lyophilization according to additional process step d) may, in specific embodiments, be performed in the presence of a disaccharide selected from the group consisting of saccharose, saccharose, lactose and trehalose, wherein the at least one disaccharide is present in an amount of at least 40 wt.-% with regard to the total weight of the lyophilized composition, said disaccharide is not added to the mixture received from process step c) but to the initial mixture as provided in process step a).

In a third aspect, the present invention provides a lyophilized pharmaceutical composition comprising an inhalable immunosuppressive macrocyclic active ingredient, specifically CsA, in liposomally solubilized form for reconstitution in an aqueous carrier liquid that may be obtained or is obtainable by the process according to the second aspect of the invention. In specific embodiments, such lyophilized pharmaceutical composition may comprise
an inhalable immunosuppressive macrocyclic active ingredient, preferably CsA;
a membrane-forming substance selected from the group of phospholipids;
a solubility-enhancing substance selected from the group of non-ionic surfactants; and
optionally one or more excipients.

In specific embodiments and dependent on the amounts of the above-mentioned ingredients used, the lyophilized pharmaceutical compositions that may be prepared according to the process of the second aspect of the invention may comprise an inhalable immunosuppressive macrocyclic active ingredient, specifically CsA, in an amount in the range of from about 2 to about 4 wt.-%, preferably of from about 2.2 to about 3.4 wt.-% or even more preferably of from about 2.4 to about 3.4 wt.-% or from about 2.4 wt.-% to about 3.0 wt.-%, or from about 2.5 wt.-% to about 2.9 wt.-% or from about 2.6 wt.-% to about 2.8 wt.-% or from about 2.65 wt.-% to about 2.75 wt.-%, in each case based on the weight of the lyophilized composition.

In further specific embodiments, the content of the membrane-forming substance selected from the group of phospholipids, preferably Lipoid S100, in the lyophilized composition may be from about 10 or 15 wt.-% to about 30 wt.-% and preferably from about 20 to about 30 wt.-%, and even more preferably from about 23 to about 27 wt.-% based on the total weight of the lyophilized composition.

In yet further specific embodiments, the content of the solubility-enhancing substance selected from the group of non-ionic surfactants may preferably be chosen in the range of from about 0.01 to about 5 wt.-%, or from about 0.1 to about 4 wt.-%, or from about 0.5 to about 3.5 wt.-%, or from about 1 to about 3 wt.-%, preferably from about 1.5 to about 2.5 wt.-%, or from about 1.6 wt.-% to about 2.3 wt.-%, or from 1.7 wt.-% to about 2.1 wt. % or from about 1.8 to about 2.0 wt. %, in each case based on the total weight of the lyophilized composition.

The lyophilized pharmaceutical composition of this third aspect of the invention that may be obtained by the process according to the second aspect of the invention may or may not further comprise residual water after lyophilization, which may be associated to the surfaces of the liposome-forming structures or which may be contained in the inner lumen of the potentially hollow liposome-forming structures as described above. In preferred embodiments, the amount of residual water comprised by the lyophilized composition is in the range of up to about 5 wt.-%, or up to about 3 wt.-%, or preferably up to about 2 wt.-%, based on the total weight of the lyophilized pharmaceutical composition.

In further specific embodiments, the lyophilized pharmaceutical composition according to the third aspect of the invention may comprise
a) liposome-forming structures comprising
   i. a therapeutically effective amount of cyclosporine A (CsA);
   ii. a membrane-forming substance selected from the group of phospholipids; and
   iii. a solubility-enhancing substance selected from the group of non-ionic surfactants;
   and
b) at least one disaccharide selected from the group consisting of saccharose, lactose and trehalose, wherein the at least one disaccharide is present in an amount of at least 40 wt.-% with regard to the total weight of the lyophilized composition.

It should be noted that in connection with this aspect of the invention also, all features, embodiments, ingredients, process parameters and combinations thereof as described in connection with the first and the second aspect of the invention apply also for this third aspect of the invention as well as for all further aspects of the invention.

In some embodiments, the at least one disaccharide is present in an amount of from at least about 40 wt.-% up to about 95 wt.-% or up to about 90 wt.-% or up to about 85 wt.-% or up to about 80 wt.-%, all with regard to the total weight of the lyophilized composition. In further specific embodiments, the lyophilisates according to this third aspect preferably comprise saccharose (sucrose) and/or lactose especially saccharose, in an amount selected in the range of from about 50 wt.-% to about 80 wt.-% or to about 75 wt.-%, with regard to or based on the total weight of the lyophilized composition. In further preferred embodiments, the lyophilized pharmaceutical compositions according to this aspect comprise the at least one disaccharide, preferably saccharose, trehalose and/or lactose, especially saccharose in an amount selected in the range of from about 60 wt.-% to about 75 wt. %, even more preferably selected in the range of from about 65 wt.-% to about 70 wt.-% with regard to the total weight of the lyophilized composition.

The lyophilized pharmaceutical composition comprising an inhalable immunosuppressive macrocyclic active ingredient, specifically CsA, in liposomally solubilized form for reconstitution in an aqueous carrier liquid, preferably comprise the inhalable immunosuppressive macrocyclic active ingredient, specifically CsA, in a therapeutically effective amount and is useful as a medicament, especially for pulmonary application by inhalation.

Accordingly, in a fourth aspect the present invention provides for a lyophilized pharmaceutical composition obtained or obtainable by a process of the second aspect of the invention for use as a medicament for pulmonary application, specifically for pulmonary application by inhalation.

In exemplary embodiments, such lyophilized pharmaceutical composition according to the third and fourth aspect of the invention comprise, or, together with further optional excipients, essentially consist or consist of, preferably comprises (each based on the total weight of the lyophilized pharmaceutical composition):

Inhalable immunosuppressive

| | | |
|---|---|---|
| macrocyclic active ingredient: | 1-7 | wt.-% |
| Disaccharide: | 40-80 | wt.-% |
| Phospholipid: | 10-40 | wt.-% |
| Non-ionic surfactant: | 0.01-10 | wt.-% |
| Buffering agent: | 1-6 | wt.-% |
| Chelating agent: | 0.05-0.5 | wt.-%, |

wherein the sum of the components adds to 100 wt.-% of the final lyophilized pharmaceutical composition.

In further exemplary embodiments, such lyophilized pharmaceutical composition according to the third and fourth aspect of the invention comprise, or, together with further optional excipients, essentially consist or consist of, preferably comprises (each based on the total weight of the lyophilized pharmaceutical composition):

| | | |
|---|---|---|
| Cyclosporine A: | 2-4 | wt.-% |
| Disaccharide: | 40-80 | wt.-% |
| Phospholipid: | 10-40 | wt.-% |
| Non-ionic surfactant: | 0.01-10 | wt.-% |
| Buffering agent: | 1-6 | wt.-% |
| Chelating agent: | 0.05-0.5 | wt.-%, |

wherein the sum of the components adds to 100 wt.-% of the final lyophilized pharmaceutical composition. It should be noted that that the values and ranges given above are calculated on the basis of a lyophilized and completely anhydrous composition. For practical reasons, however, the lyophilized composition in addition to the components listed above may or may not contain residual amounts of water in the range of from about 0 to about 5 wt.-% based on the weight of the lyophilized pharmaceutical composition.

In a further exemplary embodiment, such lyophilized pharmaceutical compositions comprise, or, together with further optional excipients, essentially consist or consist of, preferably comprises (each based on the total weight of the lyophilized pharmaceutical composition):

| | | |
|---|---|---|
| Cyclosporine A: | 2-4 | wt.-% |
| Disaccharide: | 50-75 | wt. % |
| Phospholipid: | 15-40 | wt.-% |
| Non-ionic surfactant: | 0.1-4 | wt.-% |
| Buffering agent: | 2-6 | wt.-% |
| Chelating agent: | 0.05-0.5 | wt.-%, |

wherein the sum of the components adds to 100 wt.-% of the final lyophilized pharmaceutical composition and wherein the lyophilized composition in addition to the components listed above may or may not contain residual amounts of water in the range of from about 0 to about 2 wt.-% based on the weight of the lyophilized pharmaceutical composition.

In a preferred exemplary embodiment, the lyophilized pharmaceutical composition comprises, or, together with further optional excipients, essentially consist or consist of, preferably comprises (each based on the total weight of the lyophilized pharmaceutical composition):

| | | |
|---|---|---|
| Cyclosporine A: | 2.5-3 | wt.-% |
| Disaccharide: | 60-75 | wt. % |
| Phospholipid: | 20-30 | wt.-% |
| Non-ionic surfactant: | 1-3 | wt.-% |
| Buffering agent: | 3-5 | wt.-% |
| Chelating agent: | 0.05-0.2 | wt.-%, |

wherein the sum of the components adds to 100 wt.-% of the final lyophilized pharmaceutical composition and wherein the lyophilized composition in addition to the components listed above may or may not contain residual amounts of water in the range of from about 0 to about 2 wt.-% based on the weight of the lyophilized pharmaceutical composition.

The lyophilized pharmaceutical composition according to the third and fourth aspect of the invention may be reconstituted (redispersed) in an aqueous carrier liquid, preferably in a sterile aqueous carrier liquid to form a colloidal solution or dispersion. In preferred embodiments, the pulmonary application of the lyophilized pharmaceutical composition is carried out after said reconstitution in an aqueous carrier liquid to form a colloidal solution or dispersion.

As described above, the compositions comprising an inhalable immunosuppressive macrocyclic active ingredient in liposomally solubilized form which can be prepared by the processes of the present invention in form of a dispersion or in form of a lyophilizate can be used as medicaments, especially after lyophilization and reconstitution in an aqueous carrier liquid as mentioned above, for example for the prophylaxis and treatment of autoimmune diseases, skin diseases, after transplantations or diseases of the sensory organs (eyes, nose, ear), malaise and pulmonary diseases, for example, asthma, chronic obstructive bronchitis, parenchymal, fibrotic and interstitial lung diseases or inflammations, lung cancer, and preferably for the prevention and treatment of acute or chronic transplant rejection reactions and the diseases resulting therefrom such as bronchiolitis obliterans, especially after lung, heart, bone marrow or stem cell transplantations, especially preferred after lung transplantations. It may further be used to increase the efficacy of other medicaments, in particular, of cytostatics, where an additive or synergistic effect may be achieved with cyclosporine through the efflux pump inhibition effect. Nasal, oral, ophthalmic, mucosal, parenteral or topical application of the composition according to the present invention can, in individual cases, be advantageous. The administration may be affected by application, dropping, spraying onto or into the body, which, in initial tests on humans, proved to be particularly well tolerated.

Preferably, however, the pharmaceutical compositions that can be prepared according to the present invention, especially in lyophilized and/or reconstituted form, are useful for the treatment of pulmonary diseases, in particular, asthma, refractory asthma, chronic obstructive bronchitis, parenchymal, fibrotic and interstitial lung diseases and inflammations, and preferably for the prevention and treatment of acute and chronic organ transplant rejection reactions after lung transplantations and the diseases resulting therefrom such as bronchiolitis obliterans.

As mentioned above, pharmaceutical compositions that may be prepared according to the processes of the present invention as described in detail above in connection with the first and second aspect of the invention are useful as medicaments for pulmonary application. The pulmonary application may be carried out after reconstitution or, more specifically, after redispersion of the lyophilized pharmaceutical composition that may be prepared as described above in connection with the second aspect of the invention in an aqueous carrier liquid, preferably in a sterile aqueous carrier liquid, to form a colloidal solution or dispersion, preferably to form a colloidal dispersion.

In preferred embodiments, the pulmonary application of the lyophilized pharmaceutical composition for use as described above is carried out by inhalation. In further preferred embodiments, the pulmonary application is carried out after conversion of the pharmaceutical composition comprising an inhalable immunosuppressive macrocyclic active ingredient, specifically CsA, in liposomally solubilized form into an aerosol, such as by nebulization or aerosolization. After reconstitution or, more specifically, after dispersion in an aqueous carrier liquid, the pharmaceutical compositions obtainable by the processes of the present invention may advantageously be aerosolized and administered by a nebulizer able to convert a solution, colloidal formulation or suspension such as the present compositions comprising a liposomally solubilized inhalable immunosuppressive macrocyclic active ingredient, specifically CsA, into a high fraction of droplets which are able to reach the periphery of the lungs. Practically, a jet nebulizer, ultrasonic nebulizer, piezoelectric nebulizer, electro-hydrodynamic nebulizer, membrane nebulizer, electronic membrane nebulizer, or electronic vibrating membrane nebulizer may be used. Examples of suitable nebulizers include the SideStream® (Philips), AeroEclipse® (Trudell), LC Plus® (PARI), LC Star® (PARI), LC Sprint® (PARI), I-Neb® (Philips/Respironics), IH50 (Beurer), MicroMesh® (Health & Life, Schill), Micro Air® U22 (Omron), Multisonic® (Schill), Respimat® (Boehringer), eFlow® (PARI), AeroNebGo® (Aerogen), AeroNeb Pro® (Aerogen), and AeroDose® (Aerogen) device families.

Preferably however, a piezoelectric nebulizer, electro-hydrodynamic nebulizer, membrane nebulizer, electronic membrane nebulizer, or electronic vibrating membrane nebulizer may be used. In these cases, suitable nebulizers comprise the I-Neb® (Philips/Respironics), IH50 (Beurer), MicroMesh® (Health & Life, Schill), Micro Air® U22 (Omron), Multisonic® (Schill), Respimat® (Boehringer), eFlow® (PARI), AeroNebGo® (Aerogen), AeroNeb Pro® (Aerogen), and AeroDose® (Aerogen) device families.

In preferred embodiments, the pulmonal application of the pharmaceutical composition comprising an inhalable immunosuppressive macrocyclic active ingredient, specifically CsA, in reconstituted form for use according to this aspect of the invention is carried out by means of an ultrasonic or electronic vibrating membrane nebulizer, preferably by means of a vibrating membrane nebulizer such as, for example, a device of the eFlow®, AeroNeb Pro or -Go or I-Neb type.

In further preferred embodiments, for targeting the drug CsA, especially in liposomally solubilized form as described above, to the lower respiratory tract, the composition for use according to this aspect of the present invention is aerosolized with an electronic vibrating membrane nebulizer. In a particularly preferred embodiment, the lyophilized pharmaceutical composition in reconstituted form for use according to the present invention is aerosolized with an eFlow® nebulizer (PARI Pharma GmbH).

The eFlow® nebulizer nebulizes liquid drug formulations, such as the pharmaceutical compositions that may be prepared by the processes of the present invention in reconstituted form, with a perforated vibrating membrane resulting in an aerosol with a low ballistic momentum and a high percentage of droplets in a respirable size range, usually below 5 μm. The eFlow® is designed for a more rapid and efficient nebulization of medication due to a higher nebulization rate, lower drug wastage and a higher percentage of drug available as delivered dose (DD) and respirable dose (RD) compared to conventional nebulizers such as jet nebulizers.

As described above, the pharmaceutical composition comprising an inhalable immunosuppressive macrocyclic active ingredient, specifically CsA, that may be prepared according to the processes of the present invention may be dispersed (reconstituted) in an aqueous carrier liquid, such as water or saline, preferably saline (aqueous sodium chloride solution with a concentration of 0.25% w/v), to provide an opalescent dispersion or solution. In further embodiments, such liquid liposomal dispersions are essentially free from visible particles. The liposomes comprised by said dispersion preferably have an average diameter or, more specifically, a z-average diameter of at most about 100 nm as measured by photon correlation spectroscopy using a Malvern ZetaSizer. Preferably, the liquid liposomal dispersion comprises liposomes with a z-average diameter as measured by photon correlation spectroscopy (Malvern ZetaSizer) in the range of from about 40 nm to about 100 nm and even more preferably in the range of from about 40 nm to about 70 nm.

In further specific embodiments, said liquid liposomal dispersions have a polydispersity index (PI) as measured by photon correlation spectroscopy of up to about 0.50, preferably of up to about 0.4 and even more preferably in the range of from about 0.1 to about 0.3.

In further embodiments, the liquid liposomal dispersions as described above may have an osmolality in the range of from about 300 to about 550 mOsmol/kg, preferably in the range of from about 430 to about 550 mOsmol/kg or from about 370 to about 470 mOsmol/kg. The pH-value of the liquid liposomal dispersions according to this aspect of the invention preferably is in the range of from about 6.0 to 7.0, preferably in the range of from about 6.2 to about 6.8. In further embodiments, after 1:10 dilution, the liquid liposomal dispersion according to this aspect of the invention has a turbidity of up to 200 NTU (Nephelometric Turbidity Units), preferably within the range of from about 55 to about 90 NTU.

It has surprisingly been found that the liquid liposomal dispersions of the pharmaceutical composition comprising an inhalable immunosuppressive macrocyclic active ingredient, specifically CsA, that may be prepared by reconstitution of the lyophilized pharmaceutical compositions obtainable by the process of the second aspect of the invention, especially which have been prepared by redispersing a lyophilized pharmaceutical composition of the third aspect of the present invention comprising a disaccharide selected from the group consisting of saccharose, lactose and trehalose, in an amount of at least 40 wt.-% in an aqueous carrier liquid, comprise liposomes that are equal in size or only slightly larger compared to the liposomes in a corresponding dispersion prior to the lyophilization as described below. Accordingly, the processes of present invention provide—after lyophilization and reconstitution in an aqueous carrier liquid—liquid liposomal aqueous dispersions comprising liposomes with a median diameter measured as the z-average diameter as measured by photon correlation spectroscopy (Malvern ZetaSizer) which is equal or up to 20% larger, preferably only up to 10% larger than the z-average diameter of the liposomes used to prepare the lyophilized pharmaceutical composition of the present invention prior to lyophilization, preferably which is equal or up to 20% larger than the liposomes formed by a process according to the second aspect of the invention before lyophilization.

Furthermore, it has been surprisingly found that the process according to the first aspect of the invention allows for the preparation of a dispersion comprising an inhalable macrocyclic active ingredient, specifically cyclosporine A, in liposomally solubilized form with a precise content of the active ingredient correlating to the amount of said active ingredient as introduced to the process, especially in cases in which an intermediate filtration step is involved. Accordingly, in specific embodiments the content of the inhalable macrocyclic active ingredient, specifically cyclosporine A, in liposomally solubilized form comprised by the dispersion prepared according to the process of the first aspect of the present invention comprises at least about 95% or at least about 97% or at least about 98%, such as from about 98% or from about 99% to about 100%, or from about 98% to about 99.95% or from about 98.5% to about 99.9% of the amount of the inhalable macrocyclic active ingredient, specifically cyclosporine A, as provided in the in the initial mixture according to step a) of the process of the first aspect of the present invention.

The following is a list of numbered embodiments comprised by the present invention:
1. Process for the preparation of a dispersion comprising an inhalable immunosuppressive macrocyclic active ingredient in liposomally solubilized form in an aqueous carrier liquid, the process comprising the steps of
   a) providing a mixture comprising
      the inhalable immunosuppressive macrocyclic active ingredient;
      a membrane-forming substance selected from the group of phospholipids;
      a solubility-enhancing substance selected from the group of non-ionic surfactants;
      optionally one or more excipients; and
      the aqueous carrier liquid;
   b) dispersing the mixture as provided in step a) to form an intermediate aqueous dispersion comprising the inhalable immunosuppressive macrocyclic active ingredient in the aqueous carrier liquid; and
   c) homogenizing the intermediate aqueous dispersion as formed in step b) to form the dispersion comprising the inhalable immunosuppressive macrocyclic active ingredient in liposomally solubilized form.
2. The process according to item 1, wherein dispersing according to step b) is conducted using a rotor-stator-type disperser.
3. The process according to item 1 or 2, wherein the dispersing according to step b) is performed at a shear rate (shear velocity) of at least 22,000 1/s.
4. The process according to any one of the preceding items, wherein the dispersing according to step b) is conducted using an immersion disperser.
5. The process according to any one of the preceding items, wherein the dispersing according to step b) is performed at a shear rate (shear velocity) selected within the range of from about 25,000 1/s to about 40,000 1/s.
6. The process according to any one of the proceeding items, wherein the dispersing according to step b) is performed at a shear frequency of at least 42,000 1/s.
7. The process according to any one of the preceding items, wherein the dispersing according to step b) is performed at a shear frequency selected within the range of from about 50,000 1/s to about 80,000 1/s.
8. The process according to any one of the preceding items, wherein the dispersing according to step b) is performed at a shear rate selected within the range of from about 28,000 1/s to about 37,000 1/s and at a shear frequency selected within the range of from about 50,000 1/s to about 80,000 1/s.
9. The process according to any one of items 2 to 8, wherein the rotor has a diameter within the range of from about 60 mm to about 140 mm.
10. The process according to any one of items 2 to 9, wherein the rotor has a multiplicity of teeth selected within the range of from about 10 to about 40 teeth.
11. The process according to any one of items 2 to 10, wherein the stator has a multiplicity of teeth selected within the range of from about 10 to about 40 teeth.
12. The process according to any one of items 2 to 11, wherein the rotor and/or the stator has multiple rows of teeth.
13. The process according to any one of items 2 to 12, wherein the rotor and/or the stator has 2 to 8 rows of teeth, or 3 to 6 rows of teeth.
14. The process according to any one of items 2 to 13, wherein the dispersing according to step b) is performed in a vessel having a diameter within the range of from about 700 mm to about 1,000 mm.
15. The process according to any one of items 2 to 14, wherein the dispersing according to step b) is conducted at a rotational speed within the range of from about 2,000 to about 6,000 rpm.
16. The process according to any one of items 2 to 15, wherein the dispersing according to step b) is conducted at a rotational speed within the range of from about 3,000 to about 5,500 rpm.
17. The process according to any one of items 1 to 3, wherein dispersing according to step b) is conducted using an inline disperser.
18. The process according to item 17, wherein the dispersing according to step b) is performed at a shear rate (shear velocity) selected within the range of from about 45,000 1/s to about 90,000 1/s.
19. The process according to item 16 or 17, wherein the dispersing according to step b) is performed at a shear frequency selected within the range of from about 100,000 1/s to about 200,000 1/s.
20. The process according to any one of items 17 to 19, wherein the dispersing according to step b) is performed at a shear frequency selected within the range of from about 120,000 1/s to about 180,000 1/s.
21. The process according to any one of items 17 to 20, wherein the dispersing according to step b) is performed at a shear rate selected within the range of from about 55,000 1/s to about 85,000 1/s and at a shear frequency selected within the range of from about 130,000 1/s to about 170,000 1/s.
22. The process according to any one of items 17 to 21, wherein the rotor has a diameter within the range of from about 100 mm to about 140 mm.
23. The process according to any one of items 17 to 22, wherein the rotor has a multiplicity of teeth selected within the range of from about 30 to about 50 teeth.
24. The process according to any one of items 17 to 23, wherein the stator has a multiplicity of teeth selected within the range of from about 30 to about 80 teeth.
25. The process according to any one of items 17 to 24, wherein the rotor and/or the stator has multiple rows of teeth.
26. The process according to any one of items 17 to 25, wherein the rotor and/or the stator has 3 to 12 rows of teeth, or 6 to 10 rows of teeth combined.
27. The process according to any one of items 17 to 26, wherein the dispersing according to step b) is conducted at a rotational speed within the range of from about 2,000 to about 6,000 rpm.
28. The process according to any one of items 17 to 27, wherein the dispersing according to step b) is conducted at a rotational speed within the range of from about 3,500 to about 4,500 rpm.
29. The process according to any one of items 2 to 28, wherein the dispersing according to step b) is conducted with a circumferential speed of the rotor of at least 10 m/s.
30. The process according to any one of items 2 to 29, wherein the dispersing according to step b) is conducted with a circumferential speed of the rotor selected within the range of from about 15 m/s to about 40 m/s.
31. The process according to any one of the preceding items, wherein the dispersing according to step b) is conducted using a disperser with a motor having a power of at least 2 kW.
32. The process according to any one of the preceding items, wherein the dispersing according to step b) is conducted using a disperser with a motor having a power selected within the range of from about 2 kW to about 10 kW.
33. The process according any one of the preceding items, wherein the dispersing according to step b) is conducted using an immersion disperser and an inline disperser.
34. The process according to item 33, wherein the immersion disperser and the inline disperser are used consecutively.
35. The process according to item 33 or 34, wherein the dispersing is started using an immersion disperser followed by an inline disperser.
36. The process according to any one of the preceding items, wherein the dispersing according to step b) is performed for a period of at least about 3 h.
37. The process according to any one of the preceding items, wherein the dispersing according to step b) is performed for a period within the range of from about 3 h to about 8 h.
38. The process according to any one of the preceding items, wherein the dispersing according to step b) is performed for a period within the range of from about 3 h to about 5 h.
39. The process according to any one of items 33 to 38, wherein the dispersing according to step b) is conducted using an immersion disperser for a period of up to about 1 hour followed by dispersing using an inline disperser for a period of from about 1 h to about 4 h.
40. The process according to any one of the preceding items, wherein the dispersing according to step b) is performed at a temperature (of the mixture) selected within the range of from about 15° C. to about 30° C.
41. The process according to any one of the preceding items, wherein the dispersing according to step b) is performed at ambient pressure.
42. The process according to any one of the preceding items, wherein the process comprises as a further step
   b1) filtrating of the intermediate aqueous dispersion comprising the inhalable immunosuppressive macrocyclic active ingredient in the aqueous carrier liquid as formed in step b) prior to homogenizing the resulting filtered intermediate aqueous dispersion according to step c).
43. The process according to item 41, wherein the filtration according to step b1) is performed using a filter with a mean pore width within the range of from about 200 μm to about 250 μm.
44. The process according to any one of the preceding items, wherein the inhalable immunosuppressive macrocyclic active ingredient is cyclosporine A (CsA) or tacrolimus.
45. The process according to any one of the preceding items, wherein the inhalable immunosuppressive macrocyclic active ingredient is cyclosporine A (CsA).
46. The process according to any one of the preceding items, wherein the mixture provided in step a) comprises the inhalable immunosuppressive macrocyclic active ingredient, specifically cyclosporine A (CsA), in a concentration selected within the range of from about 1 g/L to about 7 g/L, or from about 2 g/L to about 6 g/L, or from about 3 to about 5 g/L, for example 4 g/L.
47. The process according to anyone of the preceding items, wherein the membrane-forming substance selected from the group of phospholipids is a mixture of natural phospholipids.
48. The process according to any one of the preceding items, wherein the membrane-forming substance selected from the group of phospholipids is a lecithin containing unsaturated fatty acid residues.
49. The process according to any one of the preceding items, wherein the membrane-forming substance selected from the group of phospholipids is a lecithin selected from the group consisting of soybean lecithin, Lipoid S75, Lipoid S100, Phospholipon®G90, 100 or a comparable lecithin.
50. The process according to any one of the preceding items, wherein the mixture provided in step a) comprises the membrane-forming substance selected from the group of phospholipids in a concentration selected within the range of from about 20 g/L to about 60 g/L, or from about 30 g/L to about 50 g/L, or from about 30 g/L to about 40 g/L.
51. The process according to any one of the preceding items, wherein the solubility-enhancing substance selected from the group of non-ionic surfactants is selected from the group of polysorbates.
52. The process according to any one of the preceding items, wherein the solubility-enhancing substance selected from the group of non-ionic surfactants is polysorbate 80.
53. The process according to any one of the preceding items, wherein the mixture provided in step a) comprises the solubility-enhancing substance selected from the group of non-ionic surfactants in a concentration selected about 1 g/L to about 5 g/L, or from about 2 g/L to about 4 g/L, or from about 2.5 g/L to about 3.5 g/L.
54. The process according to any one of the preceding items, wherein the weight ratio of phospholipid to polysorbate is selected in the range of from 15:1 to 9:1, preferably between from about 14:1 to about 12:1, for example, about 13:1.
55. The process according to any preceding item, wherein the weight ratio between the (sum of the) phospholipid and the nonionic surfactant on the one hand and the inhalable immunosuppressive macrocyclic active ingredient, specifically CsA, on the other hand is selected in the range of from about 5:1 to about 20:1, preferably from about 8:1 to about 12:1 and more preferably about 9:1.
56. The process according to any one of the preceding items, wherein the weight ratio between the phospholipid (lecithin), the nonionic surfactant and the inhalable immunosuppressive macrocyclic active ingredient, specifically CsA, is between about 15:1:1.5 and 5:0.3:0.5, and preferably at about 9:0.7:1.
57. The process according to any one of the preceding items, wherein the inhalable immunosuppressive macrocyclic active ingredient, specifically CsA, is at least partially incorporated (or intercalated) in the bilayer membrane of the liposome-forming structures.
58. The process according to any one of the preceding items, wherein the inhalable immunosuppressive macrocyclic active ingredient, specifically CsA, is predominantly (for example by at least about 90% or even at least about 95% to about 97.5%) incorporated in the bilayer membrane of the liposome-forming structures.
59. The process according to any one of the preceding items, wherein at least about 90% or about 95% to about 97.5% or to about 99% or 99.5% or even 99.9% of the total amount of the inhalable immunosuppressive macrocyclic active ingredient, specifically CsA, is incorporated in the bilayer membranes of the liposome-forming structures formed in the process of the present invention.
60. The process according to any of the preceding items, wherein the mixture provided according to step a) comprises at least one excipient selected from the group consisting of sugars, buffers and chelating agents.
61. The process according to any one of the preceding items, wherein the mixture provided according to step a) comprises at least one disaccharide as an excipient.
62. The process according to any one of the preceding items, wherein the mixture provided according to step a) comprises at least one disaccharide selected from the group consisting of saccharose (sucrose), lactose and trehalose as an excipient.
63. The process according to any one of the preceding items, wherein the mixture provided according to step a) comprises at least one disaccharide selected from the group consisting of saccharose (sucrose), lactose and trehalose as an excipient in a concentration selected within the range of 60 g/L to about 140 g/L, or from about 80 g/L to about 120 g/L, or from about 90 g/L to about 110 g/L.
64. The process according to any one of the preceding items, wherein the mixture provided according to step a) comprises saccharose.
65. The process according to any one of the preceding items, wherein the aqueous carrier liquid comprises an aqueous sodium chloride solution (saline) or sterilized water.
66. The process according to any one of the preceding items, wherein the step of homogenizing the intermediate aqueous dispersion formed in step b) to form the dispersion comprising the inhalable immunosuppressive macrocyclic active ingredient in liposomally solubilized form comprises high pressure homogenisation.
67. The process according to item 66, wherein the high-pressure homogenisation is conducted at a pressure selected within the range of from about 100 to about 1,000 bar.
68. The process according to item 66 or 67, wherein the high-pressure homogenisation according to step c) is conducted using a piston-gap-type homogeniser.
69. A process for the preparation of a lyophilized pharmaceutical composition for reconstitution in an aqueous carrier liquid, the lyophilized pharmaceutical composition comprising an inhalable immunosuppressive macrocyclic active ingredient in liposomally solubilized form,
   wherein the process comprises the preparation of a dispersion comprising an inhalable immunosuppressive macrocyclic active ingredient in liposomally solubilized form in an aqueous carrier liquid according to the process of any one of the preceding items; and further comprising the step of
   d) removing the aqueous carrier liquid at least partially under lyophilization conditions to form the lyophilized pharmaceutical composition.
70. The process according to item 69, wherein the lyophilized pharmaceutical composition comprising an inhalable immunosuppressive macrocyclic active ingredient in liposomally solubilized form comprises
   an inhalable immunosuppressive macrocyclic active ingredient;
   a membrane-forming substance selected from the group of phospholipids;
   a solubility-enhancing substance selected from the group of non-ionic surfactants; and
   optionally one or more excipients.
71. A lyophilized pharmaceutical composition comprising an inhalable immunosuppressive macrocyclic active ingredient in liposomally solubilized form for reconstitution in an aqueous carrier liquid, wherein the composition is obtained or obtainable by a process according to item 69 or 70.
72. The lyophilized pharmaceutical composition according to item 71, comprising
   a) liposome-forming structures comprising
      a therapeutically effective amount of cyclosporine A (CsA);
      a membrane-forming substance selected from the group of phospholipids; and
      a solubility-enhancing substance selected from the group of non-ionic surfactants;
   and
   b) at least one disaccharide selected from the group consisting of saccharose, lactose and trehalose,
   wherein the at least one disaccharide is present in an amount of at least 40 wt-% with regard to the total weight of the lyophilized composition.
73. The lyophilized pharmaceutical composition of item 71 or 72 for use as a medicament for pulmonary application.
74. The lyophilized pharmaceutical composition for use according to item 73, wherein the pulmonary application is carried out after reconstitution (dispersion) of the lyophilized pharmaceutical composition of items 71 or 72 in an aqueous carrier liquid to form a colloidal solution or dispersion.
75. The lyophilized pharmaceutical composition for use according to item 73 or 74, wherein the pulmonary application is carried out after conversion of the composition into an aerosol, such as by nebulization.
76. The lyophilized pharmaceutical composition for use according to any of items 73 to 75, wherein the pulmonary application is carried out by inhalation.
77. The lyophilized pharmaceutical composition for use according to any of items 73 to 76, wherein the pulmonal application is carried out by means of an ultrasonic or electronic vibrating membrane nebulizer, preferably by means of a vibrating membrane nebulizer such as, for example, a device of the eFlow®, AeroNeb Pro or -Go or I-Neb type.
78. The lyophilized pharmaceutical composition for use according to any of items 73 to 77 for the prophylaxis and treatment of asthma, refractory asthma, chronic obstructive bronchitis, parenchymal, fibrotic and interstitial lung diseases and inflammations, and preferably for the prevention and treatment of acute and chronic organ transplant rejection reactions after lung transplantations and the diseases resulting therefrom such as bronchiolitis obliterans.

The following examples serve to illustrate the present invention without, however, limiting it in any respect:

### EXAMPLES

Example 1: Preparation of a dispersion comprising cyclosporine A in liposomally solubilized form

1.1 Step a: Preparation of the initial ingredient mixture:

1.1.1 Approximately 70% (˜104 L) water for injections was filled into the preparation vessel. It was degassed by introduction of nitrogen gas and tempered to a temperature of 40 to 45° C. 18.0 kg of saccharose, 450.0 g of sodium dihydrogen phosphate dihydrate, 612.0 g of disodium hydrogen phosphate decahydrate and 36.0 g of disodium edetate were added together and approximately 5% (8.0 L) of water for injections were used for rinsing. The mixture was stirred until a visually clear solution was obtained.

1.1.2 The solution was cooled down to 5 to 25° C. and 6480.0 g of soybean lecithin Lipoid S100 was added and stirred until a homogenous mixture was obtained. Then, 504.0 g of polysorbate 80 HP (Tween 80) was added under gentle stirring to avoid foaming and the container holding the polysorbate was rinsed with approximately 100 mL of water for injections. After that, 720.0 g of cyclosporine and approximately 5% (8 L) of water for injections was added.

1.2 Step b: Dispersion of the initial ingredient mixture

1.2.1 The ingredient mixture as prepared according step 1.1 above was then transferred into a vessel with a capacity of 400 L and dispersed for 8 h at a rotational speed of 4.800 rpm using a Ultra Turrax® UTE 115-P (IKA, Germany) high shear immersion disperser equipped with a cylindrical stator with an inner diameter of 102.5 mm and 30 teeth and a rotor (TP4, IKA, Germany) having an outer diameter of 101.0 mm and 27 teeth (gap between rotor teeth as well as between stator teeth: 4.0 mm) with a motor power of 5.5 kW resulting in a shear rate of 33,828 1/s and a shear frequency of 64,800 1/s until a homogenous dispersion was formed. After that, the resulting dispersion was stirred for 3 hours and following that, 2 portions of 10 L (each) of water for injection were added to reduce foam generation.

1.3 Step c: Homogenization of the intermediate aqueous dispersion

1.3.1 Following that, the resulting dispersion was transferred to GEA high pressure homogenizer via a stainless-steel protection filter with a pore size of 225 μm (Rigimesh® filter, PALL) and then exposed to high-pressure homogenization at a pressure of 100 bar (first stage) and 1,000 bar (second stage), respectively, at a temperature of up to 25° C. The high-pressure homogenization was repeated 9 times (cycles). After the eighth homogenization approx. 8 L of water for injections were added.

1.4 Step d: Bioburden reduction

1.4.1 The resulting homogenized dispersion was then filtered through a bioburden reduction filter with a pore size of 0.2 μm (Fluorodyne® EX; PALL) and transferred into a filling/storage tank.

1.5. Product characterization

1.5.1 The resulting homogenized dispersion had a cyclosporine A content of 100% of the total amount of cyclosporine A added in step 1.1.2.

Example 2: Preparation of a dispersion comprising cyclosporine A in liposomally solubilized form

2.1 Step a: Preparation of the initial ingredient mixture:

2.1.1 The preparation of the initial ingredient mixture as outlined under item 1.1 above was exactly repeated.

2.2 Step b: Dispersion of the initial ingredient mixture

2.2.1 The ingredient mixture as prepared according step 2.1 above was then transferred into a vessel with a capacity of 400 L and dispersed for 80 min at a rotational speed of 3,000 rpm using a Ultra Turrax® UTE 115-P (IKA, Germany) high shear immersion disperser equipped with a cylindrical stator with an inner diameter of 102.5 mm and 30 teeth and a rotor (TP4, IKA, Germany) having an outer diameter of 101.0 mm and 27 teeth (gap between rotor teeth as well as between stator teeth: 4.0 mm) with a motor power of 5.5 kW resulting in a shear rate of 21,153 1/s and a shear frequency of 40,500 1/s until a homogenous dispersion was formed. After that, the resulting dispersion was stirred for 3 hours and following that, 2 portions of 10 L (each) of water for injection were added to reduce foam generation.

2.3 Step c: Homogenization of the intermediate aqueous dispersion

2.3.1 Following that, the resulting dispersion was transferred to GEA high pressure homogenizer via a protection filter with a pore size of 40 μm (40 μm HDC II filter (all-polypropylene), PALL) and then exposed to high-pressure homogenization at a pressure of 100 bar (first stage) and 1,000 bar (second stage), respectively, at a temperature of 25° C. The high-pressure homogenization was repeated 9 times (cycles). After the eighth homogenization approx. 8 L of water for injections were added.

2.4 Step d: Bioburden reduction

2.4.1 The resulting homogenized dispersion was then filtered through a bioburden reduction filter with a pore size of 0.2 μm (Fluorodyne® EX; PALL) and transferred into a filling/storage tank.

2.5 Product characterization

2.5.1 The resulting homogenized dispersion had a cyclosporine content of 95.47% of the total amount of cyclosporine A added in step 2.1.

Example 3: Preparation of a dispersion comprising cyclosporine A in liposomally solubilized form using an inline disperser

3.1 Step a: Preparation of the initial ingredient mixture:

3.1.1 The preparation of the initial ingredient mixture as outlined under item 1.1 above was exactly repeated.

3.2 Step b: Dispersion of the initial ingredient mixture

3.2.1 The ingredient mixture as prepared according step 3.1 above was then transferred into a vessel with a capacity of 400 L and dispersed for 4 h at a rotational speed of 4,000 rpm using a Ultra Turrax® UTL 1000/10 (IKA, Germany) high shear inline disperser equipped with a dispersing tool (8SF, IKA, Germany) having a stator with an inner diameter of 120.1 mm and 54 teeth and a tooth gap of 1.6 mm and a rotor having an outer diameter of 119.4 mm and 42 teeth with a tooth gap of 2.0 mm with a motor power of 7.5 kW resulting in a shear rate of 71,413 1/s and a shear frequency of 151,200 1/s until a homogenous dispersion was formed. After that, the resulting dispersion was stirred for 3 hours and following that, 2 portions of 10 L (each) of water for injection were added to reduce foam generation.

3.3 Step c: Homogenization of the intermediate aqueous dispersion

3.3.1 The homogenization of the intermediate aqueous dispersion received in step

3.2.1 was exactly repeated as described in item 1.3.1 above.

3.4 Step d: Bioburden reduction

3.4.1 The resulting homogenized dispersion was then filtered through a bioburden reduction filter with a pore size of 0.2 μm (Fluorodyne® EX; PALL) and transferred into a filling/storage tank.

3.5. Product characterization

3.5.1 The resulting homogenized dispersion had a cyclosporine A content of 100% of the total amount of cyclosporine A added in step 3.1.

Example 4: Preparation of a dispersion comprising cyclosporine A in liposomally solubilized form using an immersion disperser and an inline disperser

4.1 Step a: Preparation of the initial ingredient mixture:

4.1.1 The preparation of the initial ingredient mixture as outlined under item 1.1 above is exactly repeated.

4.2 Step b: Dispersion of the initial ingredient mixture

4.2.1 The ingredient mixture as prepared according step 4.1 above is then transferred into a vessel with a capacity of 400 L and dispersed for 30 min at a rotational speed of 4.800 rpm using a Ultra Turrax® UTE 115-P (IKA, Germany) high shear immersion disperser equipped with a cylindrical stator with an inner diameter of 102.5 mm and 30 teeth and a rotor (TP4, IKA, Germany) having an outer diameter of 101.0 mm and 27 teeth (gap between rotor teeth as well as between stator teeth: 4.0 mm) with a motor power of 5.5 kW resulting in a shear rate of 33,828 1/s and a shear frequency of 64,800 1/s. Following that, the resulting mixture is dispersed for 4 h at a rotational speed of 4,000 rpm using a Ultra Turrax® UTL 1000/10 (IKA, Germany) high shear inline disperser equipped with a dispersing tool (8SF, IKA, Germany) having a stator with an inner diameter of 120.1 mm and 54 teeth and a tooth gap of 1.6 mm and a rotor having an outer diameter of 119.4 mm and 42 teeth with a tooth gap of 2.0 mm with a motor power of 7.5 kW resulting in a shear rate of 71,413 1/s and a shear frequency of 151,200 1/s until a homogenous dispersion was formed. After that, the resulting dispersion was stirred for 3 hours and following that, 2 portions of 10 L (each) of water for injection were added to reduce foam generation.

4.3 Step c: Homogenization of the intermediate aqueous dispersion

4.3.1 Following that, the resulting dispersion is transferred to GEA high pressure homogenizer via a stainless-steel protection filter with a pore size of 225 μm (Rigimesh® filter, PALL) and then exposed to high-pressure homogenization at a pressure of 100 bar (first stage) and 1000 bar (second stage), respectively, at a temperature of up to 25° C. The high-pressure homogenization was repeated 8 times (cycles). After the sixth homogenization approx. 8 L of water for injections is added.

4.4 Step d: Bioburden reduction 4.4.1 The resulting homogenized dispersion is then filtered through a bioburden reduction filter with a pore size of 0.2 μm (Fluorodyne® EX; PALL) and transferred into a filling/storage tank.

4.5. Product characterization 4.5.1 The resulting homogenized dispersion has a cyclosporine A content of 100% of the total amount of cyclosporine A added in step 4.1.

### Example 5: Aseptic Filling, Lyophilization and Packaging

5.1 Glass vials with a filling volume of 10 mL were sterilized in a hot-air sterilizing tunnel, cooled down and filled with aliquots of 1.35 mL (5 mg dosage) of the dispersion as prepared according to Example 1 as described above after aseptic sterilisation using two sterile filters with a pore size of 0.2 μm between the filling/storage tank and the filling needles. The vials were then partially closed with sterilized lyophilization stoppers and loaded into a lyophilizer (GEA Lyovac FCM) and were lyophilized according to a 72 h lyophilization cycle.

5.2 After completion of lyophilization, the vials were automatically fully stoppered in the lyophilization chamber. The vials were unloaded and closed with flip-tear-off caps. Each vial contained approximately 190 mg of an almost white, homogenous, porous lyophilization cake containing 5 mg of cyclosporine A in liposomally solubilized form with a maximum residual moisture of 2% (w/w) and a shelf life of 3 years.

5.3 The composition of the lyophilized drug product prepared as described above is summarized in Table 1 below:

TABLE 1

| | | |
|---|---|---|
| | Quantity | Quantity % |
| Ingredient | per unit | (w/w) |

| | | | |
|---|---|---|---|
| Cyclosporine A | 5 | mg | 2.69 |
| Polysorbate 80 | 3.5 | mg | 1.88 |
| Lipoid S100 | 45 | mg | 24.18 |
| Sucrose | 125 | mg | 67.16 |
| Sodium dihydrogen | 3.125 | mg | 1.68 |
| phosphate dihydrate | | | |
| Disodium hydrogen | 4.25 | mg | 2.28 |
| phosphate dodecahydrate | | | |
| Disodium edetate | 0.25 | mg | 0.13 |
| dihydrate | | | |
| Total | 186.125 | mg | 100.00 |

### Example 6: Reconstitution of the Lyophilized Composition Comprising Cyclosporine a to Yield a Colloidal Solution of Liposomally Solubilized Cyclosporine a for Nebulization and Inhalation

6.1 To an aliquot of 186.1 mg of the lyophilization cake as prepared according to Example 5 above containing 5 mg of cyclosporine A was added 1.20 ml of a sterile aqueous sodium chloride solution with a concentration of 0.25% (w/v) to give 1.35 ml of an opalescent aqueous solution of liposomal cyclosporine A for inhalation purposes with a concentration of CsA of 4 mg/mL. The deliverable volume to the device is 1.25 ml (5 mg L-CsA).

6.2 For the preparation of a corresponding colloidal solution with a content of liposomally solubilized cyclosporine A of 10 mg, an aliquot of 372.3 mg of the lyophilization cake as prepared according to Example 1 above was dissolved in 2.40 mL of a sterile aqueous sodium chloride solution with a concentration of 0.25% (w/v) to give 2.65 ml of an opalescent aqueous solution of liposomal cyclosporine A for inhalation purposes with a concentration of CsA of 4 mg/mL. The deliverable volume to the device is 2.50 ml (10 mg L-CsA).

6.3 The composition of the reconstituted drug product prepared as described above is summarized in Table 2 below:

TABLE 2

| | | |
|---|---|---|
| | Quantity | Quantity % |
| Ingredient | per unit | (w/v) |

| | | | |
|---|---|---|---|
| Cyclosporine A | 5 | mg | 0.4 |
| Polysorbate 80 | 3.5 | mg | 0.28 |
| Lipoid S100 | 45 | mg | 3.6 |
| Sucrose | 125 | mg | 10 |
| Sodium dihydrogen | 3.125 | mg | 0.25 |
| phosphate dihydrate | | | |
| Disodium hydrogen | 4.25 | mg | 0.34 |
| phosphate dodecahydrate | | | |
| Disodium edetate | 0.25 | mg | 0.02 |
| dihydrate | | | |
| Sodium chloride | 2.8 | mg | 0.22 |
| Water for Injection | Ad 1.25 | mL | Ad 100 |

### Example 7: Preparation of Lyophilized Compositions Comprising CsA in Liposomally Solubilized Form and Reconstituted Liposomal Solutions Thereof in the Presence of Lactose or Trehalose

7.1 Following the protocols of Example 1 and Example 5 above, lyophilized compositions comprising CsA in liposomally solubilized form were prepared in the presence of trehalose as the disaccharide and in the presence of lactose monohydrate. Both disaccharides were used in an amount necessary to obtain a content of the respective sugar of 7.5 and 10% (w/v) in the final reconstituted liposomal solution. Furthermore, in addition to the composition summarized in Table 2 above, corresponding liposomal solutions with a content of saccharose of 5.0 and 7.5% (w/v) were prepared. In all cases opalescent colloidal solutions were obtained with a polydispersity index (PI) and liposome diameters (measured as the z-average diameter, ZAve) as summarized in Table 3 below:

TABLE 3

| | Content | ZAve | | Opalescent |
|---|---|---|---|---|
| Disaccharide | [% (w/v)] | [nm] | PI | solution |
| Trehalose | 7.5 | 123.2 | 0.344 | Yes |
| Trehalose | 10 | 103.5 | 0.181 | Yes |
| Lactose-monohydrate | 7.5 | 89.1 | 0.115 | Yes |
| Lactose-monohydrate | 10 | 88.1 | 0.101 | Yes |
| Saccharose | 5 | 93.0 | 0.346 | Yes |
| Saccharose | 7.5 | 86.1 | 0.445 | Yes |
| Saccharose | 10 | 47.7 | 0.253 | Yes |

### Example 8: Comparison of Characteristics of Aqueous Liposomal Dispersions

comprising CsA in liposomally solubilized form before lyophilization and after reconstitution of the lyophilisate 8.1 An aqueous dispersion of liposomally solubilized CsA comprising 10% (w/v) of saccharose was prepared as described in Example 1. Likewise, an aqueous dispersion of liposomally solubilized CsA comprising 10% (w/v) of lactose was prepared. Furthermore, the aqueous dispersion comprising 10% (w/v) of saccharose was lyophilized as described in Example 5, and reconstituted using water for injections Key characteristics of the resulting dispersions are summarized in Table 4 below:

TABLE 4

| | | ZAve | | Osmolality |
|---|---|---|---|---|
| Dispersion comprising CsA | pH | [nm] | PI | [osmol/kg] |
| 10% (w/v) saccharose; before | 6.62 | 51.0 | 0.262 | 0.436 |
| lyophilization/reconstitution | | | | |
| 10% (w/v) saccharose; after | 6.64 | 55.3 | 0.230 | 0.383 |
| lyophilization/reconstitution | | | | |
| 10% (w/v) lactose; before | 6.58 | 50.5 | 0.251 | 0.433 |
| lyophilization/reconstitution | | | | |

### Example 9: Stability of the Lyophilized Compositions Comprising Liposomally

solubilized CsA; comparison of stabilities 9.1 Long-term stability of lyophilized compositions comprising cyclosporine A 9.1.1 A lyophilized pharmaceutical composition comprising cyclosporine A (5 mg) was prepared according to Example 1 and Example 5 above. The lyophilized composition in form of an almost white, homogeneous, porous lyophilization cake was aliquoted in 6R glass vials, sealed and stored at 25° C. and an air humidity of 60% relative humidity (RH) for a period of 36 months. Aliquots of the material were reconstituted with saline (0.25% (w/v)) to result in a volume 1.25 ml of the reconstituted solution before and after the storage period and the median liposome diameter (Z-average), the polydispersity index as well as the content of cyclosporine A was determined after 3 months, 6 months, 9 months 12 months 18 months, 24 months and 36 months.

9.1.2 It was found that before and after the above-described storage period all parameters were within their respective acceptance criteria. More specifically, the polydispersity index (PI) was lower or equal to 0.50 before and after each storage time period. Furthermore, the median liposome diameter (Z-average) was in the prescribed range of from 40 to 100 nm before and after each storage time period. Furthermore, the CsA content of the reconstituted solution were within the acceptance criteria in the range of from 95.0 to 105.0%. 9.1.3 The long-term stability study as described above was repeated at a temperature of 30° C. and an air humidity of 65% relative humidity (RH). All test parameters as described above were found within their acceptance criteria (as above) before and after a storage period of 3 months, 6 months, 9 months and 12 months.

9.1.4 The long-term stability study as described above was repeated using a lyophilized pharmaceutical composition comprising cyclosporine A (5 mg) prepared according to Example 1 above, wherein however, the lyophilized composition had a content of saccharose necessary to give a liquid composition with a content of saccharose of 7.5 wt.-% with regard to the total amount of the liquid composition after reconstitution.

9.1.5 In this case also, it was found that before and after the above-described storage period all parameters were within their respective acceptance criteria. More specifically, the polydispersity index (PI) was lower or equal to 0.50 before and after each storage time period. Furthermore, the median liposome diameter (Z-average) was in the prescribed range of from 40 to 100 nm before and after each storage time period. Furthermore, the CsA content of the reconstituted solution were within the acceptance criteria in the range of from 95.0 to 105.0%. 9.1.6 The experiments described under items 9.1.1 to 9.1.3 above were repeated using a lyophilized pharmaceutical composition comprising 10 mg of cyclosporine A prepared according to Examples 1 and 5 above. In this case also, it was found that before and after the above-described storage period all parameters were within their respective acceptance criteria. More specifically, the polydispersity index (PI) was lower or equal to 0.50 before and after each storage time period. Furthermore, the median liposome diameter (Z-average) was in the prescribed range of from 40 to 100 nm before and after each storage time period. Furthermore, the CsA content of the reconstituted solution were within the acceptance criteria in the range of from 95.0 to 105.0%.

### Example 10: Nebulization Experiments and Aerosol Characterization

10.1 2.5 ml (corresponding to 10 mg of CsA) of the colloidal solution as prepared in Examples 1, 5 and 6 (consecutively) were aerosolized by means of a specially adapted electronic vibrating membrane nebuliser of the PARI eFlow 30 XL type having a mixing chamber and breathing in/out valves at a flow rate of 15 L/min according to EUROPEAN PHARMACOPOEIA 7.3; 2.9.44.

10.2 The droplet size distribution of the thus produced aerosol was characterized by laser diffraction using a Malvern MasterSizer X: The mass average particle diameter thus determined was 3.3 μm (Standard Deviation (SD) 0.1) at a geometric standard deviation of 1.5. The respirable particle fraction (RF)<5 μm was 65.3% (SD 2.8), the respirable particle fraction having a particle size <3.3 μm was 37.7% (SD 2.2).

10.3 In an inhalation experiment (adult; flow rate 15 mL/min) a total amount of 9897 μg of cyclosporin A in form of a reconstituted liquid formulation as described in Example 1 above was filled in and administered with the electronic vibrating membrane nebuliser (PARI eFlow 30 XL). The delivered dose (DD) of cyclosporin A was 7339 μg (SD: 471). The respirable dose (RD)<5 μm was 6534 μg (66.0%; SD 4.3%); the RD<3.3 μm was 4461 μg (45.1%; SD 3.2%) and the respirable dose (RD)<2 μm was 1080 μg (10.9%; SD 0.9%).

### Example 11: Preparation of a Dispersion Comprising Tacrolimus in Liposomally Solubilized Form

11.1 Step a: Preparation of the initial ingredient mixture:

11.1.1 Approximately 70% (˜104 L) water for injections is filled into the preparation vessel. It is degassed by introduction of nitrogen gas and tempered to a temperature of 40 to 45° C. 18.0 kg of saccharose, 450.0 g of sodium dihydrogen phosphate dihydrate, 612.0 g of disodium hydrogen phosphate decahydrate and 36.0 g of disodium edetate are added together and approximately 5% (8.0 L) of water for injections are used for rinsing. The mixture is stirred until a visually clear solution is obtained. 11.1.2 The solution is cooled down to 5 to 25° C. and 6480.0 g of soybean lecithin Lipoid S100 is added and stirred until a homogenous mixture is obtained. Then, 504.0 g of polysorbate 80 HP (Tween 80) is added under gentle stirring to avoid foaming and the container holding the polysorbate is rinsed with approximately 100 mL of water for injections. After that, 720.0 g tacrolimus and approximately 5% (8 L) of water for injections is added.

11.2 Step b: Dispersion of the initial ingredient mixture 11.2.1 The ingredient mixture as prepared according step 11.1 above is then transferred into a vessel with a capacity of 400 L and dispersed for 8 h at a rotational speed of 4.800 rpm using a Ultra Turrax® UTE 115-P (IKA, Germany) high shear immersion disperser equipped with a cylindrical stator with an inner diameter of 102.5 mm and 30 teeth and a rotor (TP4, IKA, Germany) having an outer diameter of 101.0 mm and 27 teeth (gap between rotor teeth as well as between stator teeth: 4.0 mm) with a motor power of 5.5 kW resulting in a shear rate of 33,828 1/s and a shear frequency of 64,800 1/s until a homogenous dispersion is formed. After that, the resulting dispersion is stirred for 3 hours and following that, 2 portions of 10 L (each) of water for injection are added to reduce foam generation.

11.3 Step c: Homogenization of the intermediate aqueous dispersion

11.3.1 Following that, the resulting dispersion is transferred to GEA high pressure homogenizer via a stainless-steel protection filter with a pore size of 225 μm (Rigimesh® filter, PALL) and then exposed to high-pressure homogenization at a pressure of 100 bar (first stage) and 1,000 bar (second stage), respectively, at a temperature of up to 25° C. The high-pressure homogenization is repeated 8 times (cycles). After the sixth homogenization approx. 8 L of water for injections are added.

11.4 Step d: Bioburden reduction

11.4.1 The resulting homogenized dispersion is then filtered through a bioburden reduction filter with a pore size of 0.2 μm (Fluorodyne® EX; PALL) and transferred into a filling/storage tank.

11.5. Product characterization

11.5.1 The resulting homogenized dispersion is expected to have a tacrolimus content of 100% of the total amount of tacrolimus added in step 11.1.2.

1. Process for the preparation of a dispersion comprising an inhalable immunosuppressive macrocyclic active ingredient in liposomally solubilized form in an aqueous carrier liquid, the process comprising the steps of
a) providing a mixture comprising
the inhalable immunosuppressive macrocyclic active ingredient;
a membrane-forming substance selected from the group of phospholipids;
a solubility-enhancing substance selected from the group of non-ionic surfactants;
optionally one or more excipients; and
the aqueous carrier liquid;
b) dispersing the mixture as provided in step a) to form an intermediate aqueous dispersion comprising the inhalable immunosuppressive macrocyclic active ingredient in the aqueous carrier liquid; and
c) homogenizing the intermediate aqueous dispersion as formed in step b) to form the dispersion comprising the inhalable immunosuppressive macrocyclic active ingredient in liposomally solubilized form.

2. The process according to claim 1, wherein dispersing according to step b) is conducted using a rotor-stator-type disperser.

3. The process according to claim 1, wherein the dispersing according to step b) is performed at a shear rate (shear velocity) of at least 22,000 1/s.

4. The process according to claim 1, wherein the dispersing according to step b) is conducted using an immersion disperser.

5. The process according to claim 1, wherein the dispersing according to step b) is performed at a shear rate (shear velocity) selected within the range of from about 25,000 1/s to about 40,000 1/s.

6. The process according to claim 1, wherein the dispersing according to step b) is performed at a shear frequency selected within the range of from about 50,000 1/s to about 80,000 1/s.

7. The process according to claim 1, wherein the dispersing according to step b) is performed at a shear rate selected within the range of from about 28,000 1/s to about 37,000 1/s and at a shear frequency selected within the range of from about 50,000 1/s to about 80,000 1/s.

8. The process according to claim 2, wherein the rotor has a diameter within the range of from about 60 mm to about 140 mm.

9. The process according to claim 2, wherein the dispersing according to step b) is conducted at a rotational speed within the range of from about 2,000 to about 6,000 rpm.

10. The process according to claim 1, wherein the dispersing according to step b) is conducted using an inline disperser.

11. The process according to claim 10, wherein the dispersing according to step b) is performed at a shear rate (shear velocity) selected within the range of from about 45,000 1/s to about 90,000 1/s.

12. The process according to claim 10, wherein the dispersing according to step b) is performed at a shear frequency selected within the range of from about 100,000 1/s to about 200,000 1/s.

13. The process according to claim 10, wherein the dispersing according to step b) is performed at a shear frequency selected within the range of from about 120,000 1/s to about 180,000 1/s.

14. The process according to claim 10, wherein the dispersing according to step b) is performed at a shear rate selected within the range of from about 55,000 1/s to about 85,000 1/s and at a shear frequency selected within the range of from about 130,000 1/s to about 170,000 1/s.

15. The process according to claim 10, wherein the rotor has a diameter within the range of from about 100 mm to about 140 mm.

16. The process according to claim 10, wherein the dispersing according to step b) is conducted at a rotational speed within the range of from about 3,500 to about 4,500 rpm.

17. The process according to claim 1, wherein the dispersing according to step b) is conducted using an immersion disperser and an inline disperser.

18. The process according to claim 17, wherein the immersion disperser and the inline disperser are used consecutively.

19. The process according to claim 17, wherein the dispersing is started using an immersion-disperser followed by an inline disperser.

20. The process according to claim 2, wherein the dispersing according to step b) is conducted with a circumferential speed of the rotor selected within the range of from about 15 m/s to about 40 m/s.

21. The process according to claim 1, wherein the dispersing according to step b) is performed for a period within the range of from about 1 h to about 8 h.

22. The process according to claim 1, wherein the dispersing according to step b) is performed for a period within the range of from about 3 h to about 8 h.

23. The process according to claim 1, wherein the dispersing according to step b) is conducted using an immersion disperser for a period of up to about 1 hour followed by dispersing using an inline disperser for a period of from about 1 h to about 4 h.

24. The process according to claim 1, wherein the process comprises as a further step
b1) filtrating of the intermediate aqueous dispersion comprising the inhalable immunosuppressive macrocyclic active ingredient in the aqueous carrier liquid as formed in step b) prior to homogenizing the resulting filtered intermediate aqueous dispersion according to step c).

25. The process according to claim 24, wherein the filtration according to step b1) is performed using a filter with a mean pore width within the range of from about 200 μm to about 250 μm.

26. The process according to claim 1, wherein the process comprises as a further step
c1) sterilizing the homogenized dispersion comprising the inhalable immunosuppressive macrocyclic active ingredient, specifically cyclosporine A, in liposomally solubilized form resulting from step c).

27. The process according to claim 26, wherein the sterilizing is performed by sterile filtration.

28. The process according to claim 1, wherein the inhalable immunosuppressive macrocyclic active ingredient is cyclosporine A (CsA).

29. A process for the preparation of a lyophilized pharmaceutical composition for reconstitution in an aqueous carrier liquid, the lyophilized pharmaceutical composition comprising an inhalable immunosuppressive macrocyclic active ingredient in liposomally solubilized form,
wherein the process comprises the preparation of a dispersion comprising an inhalable immunosuppressive macrocyclic active ingredient in liposomally solubilized form in an aqueous carrier liquid according to the process of any one of the preceding claims; and further comprising the step of
d) removing the aqueous carrier liquid at least partially under lyophilization conditions to form the lyophilized pharmaceutical composition.

30. A lyophilized pharmaceutical composition comprising an inhalable immunosuppressive macrocyclic active ingredient in liposomally solubilized form for reconstitution in an aqueous carrier liquid, wherein the composition is obtained by a process according to claim 29.
